(19) United States
(12) Patent Application Publication
(10) Pub. No.: US 2024/0142448 A1
(43) Pub. Date: May 2, 2024

(54) BIOMARKER

(71) Applicant: Oxford University Innovation Limited, Oxford (GB)

(72) Inventors: Sarosh R. IRANI, Oxford (GB); Patrick WATERS, Oxford (GB)

(21) Appl. No.: 18/279,624

(22) PCT Filed: Mar. 9, 2022
(86) PCT No.: PCT/GB2022/050614

§ 371 Date: Aug. 31, 2023

(30) Foreign Application Priority Data
Mar. 9, 2022 (GB) 2103241.2

(51) Int. Cl. G01N 33/564; G01N 33/68

(52) U.S. Cl. CPC G01N 33/564; G01N 33/6854; G01N 2800/52; G01N 2800/54

(57) ABSTRACT

The invention relates to biomarkers for assessing the disease status of a subject with an antibody-associated autoimmune disease, in particular a neurological disease such as neuromyelitis optica spectrum disorders (NMOSD) or LGI1-antibody encephalitis. The invention in particular looks at the presence or levels of IgM isotype antibodies which recognise a specific antigen in an anti-body-associated autoimmune disease, which is used to determine whether a subject is in a relapse.

### TECHNICAL FIELD

The present invention relates to biomarkers and uses thereof. In particular, to biomarkers for assessing the disease status of a subject with an antibody-associated autoimmune disease, in particular a neurological disease, more specifically an antibody-mediated autoimmune disease such as neuromyelitis optica spectrum disorders (NMOSD) or LGI1-antibody encephalitis.

### BACKGROUND

Neuromyelitis optica spectrum disorders are caused by IgG autoantibodies which recognise the extracellular domain of aquaporin-4 (AQP4). The presence of IgG autoantibodies which recognise the extracellular domain of AQP4 are therefore useful in diagnosing NMOSD.

IgG autoantibodies which recognise leucine rich glioma inactivated 1 (LGI1) are used to diagnose a different illness in which patients present with seizures, cognitive deficits, peripheral nerve involvement and sleep disturbances: LGI1-antibody encephalitis (or anti-LGI1 antibody encephalitis). LGI1-autoantibodies are detected using a variety of methods including immunohistochemistry and cell-based assays. Patients with LGI1-antibody encephalitis can respond well to immunotherapies including corticosteroids, intravenous immunoglobulins, plasma exchange, B cell depleting agents (e.g. rituximab (RTX) and inebilizumab), IgG depleting agents (e.g. FcRn inhibitors), cyclophosphamide, azathioprine, mycophenolate mofetil and satralizumab. While the majority of patients improve, relapses are common, occurring at rates of around 10-40%.

AQP4-IgGs are predominantly of the IgG1 subclass, and their major pathogenic mechanism is likely via complement mediated astrocyte damage. Patient disability is accrued through discrete clinical relapses, typically of myelitis and/or optic neuritis, although other manifestations are recognised. Without treatment, patients with NMOSD will relapse on average between 1 to 5 times per year, with incremental step deteriorations in health, including accrual of disabilities, occurring with each relapse. There is also a small risk of death with attacks which affect the brainstem. Annualised relapse rates are partially reduced by a variety of immunotherapies including azathioprine, mycophenolate mofetil, eculizumab, satralizumab, inebilizumab and rituximab (RTX) (CAS 174722-31-7)—an anti-CD20 monoclonal antibody. Most patients with NMOSD are treated for life with a selection of these drugs, all of which are associated with different, sometimes serious, side effects. Therefore, a level of caution is required when prescribing such drugs, and unnecessary treatment should be avoided where possible.

RTX treatment is expensive, and is usually administered roughly every 6 months, and/or as CD19/CD27 cell counts rise. RTX effectively depletes B cells in circulation but it spares the typically CD20 negative plasma cells, and it does not consistently reduce serum AQP4-IgG levels.

There is a need in the art for additional biomarkers for determining the disease status of patients with antibody-mediated autoimmune diseases. It is an aim of the present invention therefore to provide one or more biomarker(s) that may be used to give an indication of the disease status in an individual with an antibody-mediated autoimmune disease. For example, biomarkers which may also be used to determine whether an individual with an antibody-mediated autoimmune disease is likely to, or is not likely to, benefit from treatment at a given point in time, and/or to determine whether an individual with an antibody-mediated autoimmune disease is in relapse or not.

### SUMMARY

In an aspect, there is provided a method of determining the disease status of a subject that has been diagnosed with an antibody-associated autoimmune disease wherein the antibody recognises a specific antigen, and wherein the method comprises:
a) providing a biological sample obtained from a subject; and
b) determining the level, or presence, of IgM isotype antibodies which recognise the specific antigen.

In any aspect, an antibody-associated autoimmune disease may be a neurological autoimmune disease. The antibody-associated autoimmune disease of any aspect may be selected from the group consisting of NMOSD, forms of autoimmune encephalitis such as NMDAR antibody encephalitis, pemphigus vulgaris, myasthenia gravis, Grave's disease, Type 1 diabetes, Goodpasture's syndrome, Addison's syndrome, systemic lupus erythematosus (SLE), thrombotic thrombopenic purpura, vasculitis, rheumatoid arthritis, chronic inflammatory demyelinating polyneuropathy, Sjogren's syndrome, and pernicious anaemia.

In another aspect, there is provided a method of determining the disease status of a subject that has been diagnosed with an antibody-mediated autoimmune disease wherein the antibody recognises a specific antigen, and wherein the method comprises:
a) providing a biological sample obtained from a subject; and
b) determining the level, or presence, of IgM isotype antibodies which recognise the specific antigen.

The antibody-mediated autoimmune disease of any aspect may be driven by germinal centre reactions. The antibody-mediated autoimmune disease of any aspect may be NMOSD or encephalitis.

Preferably, the antibody-mediated autoimmune disease of any aspect may be NMOSD or encephalitis.

When the antibody-associated autoimmune disease or antibody-mediated autoimmune disease is encephalitis, the specific antigen may be LGI1.

When the antibody-associated autoimmune disease or antibody-mediated autoimmune disease is NMOSD, the specific antigen may be AQP4.

When the antibody-associated autoimmune disease is pemphigus vulgaris, the specific antigen may be a Desmoglein, such as Desmoglein 1, Desmoglein 2, Desmoglein 3, Desmoglein 4.

When the antibody-associated autoimmune disease is myasthenia gravis, the specific antigen may be Acetylcholine receptor or muscle specific kinase (MuSK).

When the antibody-associated autoimmune disease is Grave's disease, the specific antigen may be thyrotropin (TSH) receptor.

When the antibody-associated autoimmune disease is type 1 diabetes, stiff person syndrome spectrum disorders, ataxia or a seizure disorder, the specific antigen may be glutamic acid decarboxylase.

When the antibody-associated autoimmune disease is Goodpastures syndrome, the specific antigen may be a collagen.

When the antibody-associated autoimmune disease is Addison's syndrome, the specific antigen may be 21-hydroxylase.

When the antibody-associated autoimmune disease is NMDAR-antibody encephalitis, the specific antigen may be the NR1 subunit of NMDA receptor.

When the antibody-associated autoimmune disease is systemic lupus erythematosus (SLE), the specific antigen may be DNA, or Smith (Sm) or ribonucleoprotein (RNP).

When the antibody-associated autoimmune disease is thrombotic thrombopenic purpura, the specific antigen may be ADAMTS13.

When the antibody-associated autoimmune disease is vasculitis, the specific antigen may be proteinase 3 and myeloperoxidase.

When the antibody-associated autoimmune disease is rheumatoid arthritis, the specific antigen may be one or more citrullinated proteins.

When the antibody-associated autoimmune disease is chronic inflammatory demyelinating polyneuropathy, the specific antigen may be one or more neurofascins, contactins or casprs.

When the antibody-associated autoimmune disease is Sjorgrens syndrome, the specific antigen may be Ro(SSA) and/or La(SSB).

When the antibody-associated autoimmune disease is pernicious anaemia, the specific antigen may expressed on a parietal cell.

In an embodiment of any aspect, when the specific antigen is LGI1, the method may additionally or alternatively comprise determining the level, or presence, of CXCL13.

Determining the disease status may include determining the likelihood of a relapse.

The presence of IgM isotype antibodies which recognise the specific antigen, and/or the presence of CXCL13, may indicate a higher likelihood of a relapse. An increase in the level of IgM isotype antibodies which recognise the specific antigen, and/or an increase in the level of CXCL13, in the biological sample obtained from a subject relative to the level of IgM isotype antibodies which recognise the specific antigen, and/or the level of CXCL13, in a reference sample may indicate a higher likelihood of a relapse. The subject may then be treated for a relapse accordingly, this may be administration of therapy, an increase in the therapy already being administered or a change in therapy.

The absence of IgM isotype antibodies which recognise the specific antigen, and/or the absence of CXCL13, may indicate a lower likelihood of a relapse. A decrease or no change in the level of IgM isotype antibodies which recognise the specific antigen, and/or a decrease or no change in the level of CXCL13, in the biological sample obtained from a subject relative to the level of IgM isotype antibodies which recognise the specific antigen, and/or the level of CXCL13, in a reference sample may indicate a lower likelihood of a relapse. The subject may then be treated accordingly, this may include no treatment is administered, no additional treatment is administered, or treatment is reduced.

Determining the disease status may include determining whether the subject is in a relapse.

The presence of IgM isotype antibodies which recognise the specific antigen, and/or the presence of CXCL13, may indicate that the subject is in a relapse. An increase in the level of IgM isotype antibodies which recognise the specific antigen, and/or an increase in the level of CXCL13, in the biological sample obtained from a subject relative to the level of IgM isotype antibodies which recognise the specific antigen, and/or the level of CXCL13, in a reference sample may indicate that the subject is in a relapse. The subject may then be treated for a relapse accordingly; this may be administration of therapy, an increase in therapy already being administered or a change in therapy.

The absence of IgM isotype antibodies which recognise the specific antigen, and/or the absence of CXCL13, may indicate that the subject is not in a relapse. A decrease in the level of IgM isotype antibodies which recognise the specific antigen, and/or a decrease or no change in the level of CXCL13, in the biological sample obtained from a subject relative to the level of IgM isotype antibodies which recognise the specific antigen, and/or the level of CXCL13, in a reference sample may indicate that the subject is not in a relapse. The subject may then be treated accordingly, this may include no treatment is administered, no additional treatment is administered, or treatment is reduced.

Determining the disease status may include determining whether the subject is to be treated, if no additional treatment is to be administered, or if treatment is to be reduced.

The presence of IgM isotype antibodies which recognise the specific antigen, and/or the presence of CXCL13, may indicate that the subject is to be treated. An increase in the level of IgM isotype antibodies which recognise the specific antigen, and/or an increase in the level of CXCL13, in the biological sample obtained from a subject relative to the level of IgM isotype antibodies which recognise the specific antigen, and/or the level of CXCL13, in a reference sample may indicate that the subject is to be treated accordingly, this may be administration of therapy, an increase in therapy already being administered or a change in therapy.

The absence of IgM isotype antibodies which recognise the specific antigen, and/or the absence of CXCL13, may indicate that the subject is not to be treated, no additional treatment is to be administered, or treatment is to be reduced. A decrease or no change in the level of IgM isotype antibodies which recognise the specific antigen, and/or a decrease or no change in the level of CXCL13, in the biological sample obtained from a subject relative to the level of IgM isotype antibodies which recognise the specific antigen, and/or to the level of CXCL13, in a reference sample may indicate that the subject is to be treated accordingly, this may include no treatment is administered, no additional treatment is administered, or treatment is reduced.

In an aspect, there is provided a method of determining the disease status of a subject that has been diagnosed with an antibody-mediated autoimmune disease, wherein the method comprises:
a) providing a biological sample obtained from a subject; and
b) determining the level, or presence, of IgM isotype antibodies which recognise AQP4; and/or the level, or presence, of IgM isotype antibodies which recognise LGI1; and/or the level, or presence, of CXCL13.

Preferably, in a), the level, or presence, of IgM isotype antibodies which recognise AQP4 is determined in a subject already diagnosed with NMOSD.

Preferably, in b), the level, or presence, of IgM isotype antibodies which recognise LGI1, and/or the level, or presence, of CXCL13, is determined in a subject already diagnosed with LGI1-encephalitis.

In another aspect, there is provided a method of determining whether a subject diagnosed with an antibody-associated autoimmune disease is in a relapse, wherein the antibody recognises a specific antigen, and wherein the method comprises:
a) providing a biological sample obtained from the subject; and
b) determining the presence or level of IgM isotype antibodies which recognise the specific antigen.

In another aspect, there is provided a method of determining whether a subject diagnosed with an antibody-mediated autoimmune disease is in a relapse, wherein the antibody recognises a specific antigen, and wherein the method comprises:
a) providing a biological sample obtained from the subject; and
b) determining the presence or level of IgM isotype antibodies which recognise the specific antigen.

The subject may be determined to be in relapse when IgM isotype antibodies which recognise the specific antigen, and/or CXCL13, are present. The subject may be determined to be in relapse when the level of IgM isotype antibodies which recognise the specific antigen, and/or the level of CXCL13, in the biological sample obtained from the subject are higher than the level of IgM isotype antibodies which recognise the specific antigen, and/or the level of CXCL13, in a reference sample. A subject who is determined to be in relapse may then be treated accordingly, this may be administration of therapy, an increase in the therapy already being administered or a change in therapy.

The subject may be determined to not be in relapse when IgM isotype antibodies which recognise the specific antigen, and/or CXCL13, are absent. The subject may be determined to not be in relapse when the level of IgM isotype antibodies which recognise the specific antigen, and/or the level of CXCL13, in the biological sample obtained from the subject are lower than, or the same as, the level of IgM isotype antibodies which recognise the specific antigen, and/or the level of CXCL13, in a reference sample. A subject who is determined not to be in relapse may then be treated accordingly, this may include no treatment is administered, no additional treatment is administered, or treatment is reduced.

In any aspect, when the antibody-mediated autoimmune disease is NMOSD, the treatment may be an anti-CD20 antibody, such as Rituximab, Ofatumumab or Ocrelizumab. Other treatments include steroids; azathioprine; mycophenolate mofetil; agents, such as mABs, which recognise IL-6R or reduce IL-6R activity; agents, such as mAbs, which recognise CD19 or reduce CD19 activity; agents which alter the function of complement pathway components (such as Eculizumab); or bortezomib; for example.

In any aspect, when the antibody-mediated autoimmune disease is encephalitis, the treatment may be with one or more immunotherapy such as with corticosteroids, intravenous immunoglobulins, plasma exchange, B cell depleting agents (e.g. rituximab (RTX) and inebilizumab), IgG depleting agents (e.g. FcRn inhibitors), cyclophosphamide, azathioprine, mycophenolate mofetil and satralizumab.

In another aspect, there is provided a method of determining whether a subject diagnosed with an antibody-mediated autoimmune disease is in a relapse, wherein the method comprises:
a) providing a biological sample obtained from the subject; and
b) determining the level, or presence, of IgM isotype antibodies which recognise AQP4; and/or the level, or presence, of IgM isotype antibodies which recognise LGI1; and/or the level, or presence, of CXCL13.

In another aspect there is provided a method of determining whether a subject diagnosed with NMOSD is in relapse, the method comprising:
a) providing a biological sample obtained from a subject; and
b) determining the presence or level of IgM isotype antibodies which recognise AQP4.

The subject may be determined to be in relapse when IgM isotype antibodies which recognise AQP4 are present. The subject may be determined to be in relapse when the level of IgM isotype antibodies which recognise AQP4 in the biological sample obtained from a subject are higher than the level of IgM isotype antibodies which recognise AQP4 in a reference sample.

A subject who is determined to be in relapse may then be treated accordingly. This may be administration of therapy, an increase in therapy already being administered or a change in therapy.

The subject may be determined to not be in relapse when IgM isotype antibodies which recognise AQP4 are absent. The subject may be determined to be in relapse when the level of IgM isotype antibodies which recognise AQP4 in the biological sample obtained from a subject are lower than or the same as the level of IgM isotype antibodies which recognise AQP4 in a reference sample. A subject who is determined not to be in relapse may then be treated accordingly, this may include no treatment is administered, no additional treatment is administered, or treatment is reduced.

In another aspect there is provided a method of determining whether a subject diagnosed with encephalitis is in relapse, the method comprising:
a) providing a biological sample obtained from a subject; and
b) determining the presence or level of IgM isotype antibodies which recognise LGI1 and/or the presence or level of CXCL13.

The encephalitis may be mediated by and/or diagnosed by the presence of IgG isotype antibodies which recognise LGI1.

The subject may be determined to be in relapse when IgM isotype antibodies which recognise LGI1, and/or CXCL13, are present. The subject may be determined to be in relapse when the level of IgM isotype antibodies which recognise LGI1, and/or the level of CXCL13, in the biological sample obtained from a subject are higher than the level of IgM isotype antibodies which recognise LGI1, and/or the level of CXCL13, in a reference sample.

A subject who is determined to be in relapse may then be treated accordingly. This may be administration of therapy, an increase in therapy already being administered or a change in therapy.

The subject may be determined to not be in relapse when IgM isotype antibodies which recognise LGI1, and/or CXCL13, are absent. The subject may be determined to be in relapse when the level of IgM isotype antibodies which recognise LGI1, and/or the level of CXCL13, in the biological sample obtained from a subject are lower than or the same as the level of IgM isotype antibodies which recognise LGI1, and/or the level of CXCL13, in a reference sample. A subject who is determined not to be in relapse may then be treated accordingly, this may include no treatment is administered, no additional treatment is administered, or treatment is reduced.

In another aspect there is provided a method of determining whether to treat a subject diagnosed with an antibody-associated autoimmune disease, wherein the antibody recognises a specific antigen, and wherein the method comprises:
a) providing a biological sample obtained from a subject; and
b) determining the presence or level of IgM isotype antibodies which recognise the specific antigen.

In another aspect there is provided a method of determining whether to treat a subject diagnosed with an antibody-mediated autoimmune disease, wherein the antibody recognises a specific antigen, and wherein the method comprises:
a) providing a biological sample obtained from a subject; and
b) determining the presence or level of IgM isotype antibodies which recognise the specific antigen; and/or presence or level or CXCL13 antigen.

It may be determined that the subject is to be treated when IgM isotype antibodies which recognise the specific antigen, and/or CXCL13, are present. It may be determined that the subject is to be treated when the level of IgM isotype antibodies which recognise the specific antigen, and/or the level of CXCL13, in the biological sample obtained from a subject are higher than the level of IgM isotype antibodies which recognise the specific antigen, and/or the level CXCL13, in a reference sample.

It may be determined when IgM isotype antibodies which recognise the specific antigen, and/or CXCL13, are absent that the subject is not to be treated further, requires no additional treatment, or can have the treatment administered reduced. When the level of IgM isotype antibodies which recognise the specific antigen, and/or the level of CXCL13, in the biological sample obtained from a subject are lower than, or the same as, the level of IgM isotype antibodies which recognise the specific antigen, and/or the level of CXCL13, in a reference sample it may be determined that the subject is not to be treated, requires no additional treatment, or can have the treatment administered reduced.

In another aspect there is provided a method of determining whether to treat a subject diagnosed with an antibody-mediated autoimmune disease, wherein the method comprises:
a) providing a biological sample obtained from a subject; and
b) determining the presence or level of IgM isotype antibodies which recognise the specific antigen; and/or the level, or presence, of one or more IgM isotype antibodies which recognise LGI1; and/or the level, or presence, of CXCL13.

In another aspect there is provided a method of determining whether to treat a subject diagnosed with NMOSD, the method comprising:
a) providing a biological sample obtained from a subject; and
b) determining the presence or level of IgM isotype antibodies which recognise AQP4.

It may be determined that the subject is to be treated when IgM isotype antibodies which recognise AQP4 are present. It may be determined that the subject is to be treated when the level of IgM isotype antibodies which recognise AQP4 are higher in the biological sample obtained from a subject than the level of IgM isotype antibodies which recognise AQP4 in a reference sample.

It may be determined that the subject is not to be treated when IgM isotype antibodies which recognise AQP4 are absent. It may be determined that the subject is not to be treated when the level of IgM isotype antibodies which recognise AQP4 in the biological sample obtained from a subject are lower than, or the same as, the level of IgM isotype antibodies which recognise AQP4 in a reference sample.

In another aspect there is provided a method of determining whether to treat a subject diagnosed with encephalitis, the method comprising:
a) providing a biological sample obtained from a subject; and
b) determining the presence or level of IgM isotype antibodies which recognise LGI1 and/or the presence or level of CXCL13.

The encephalitis may be mediated by and/or diagnosed by the presence of IgG isotype antibodies which recognise LGI1.

It may be determined that the subject is to be treated when IgM isotype antibodies which recognise LGI1, and/or CXCL13, are present. It may be determined that the subject is to be treated when the level of IgM isotype antibodies which recognise LGI1, and/or the level of CXCL13, are higher in the biological sample obtained from a subject than the level of IgM isotype antibodies which recognise LGI1, and/or the level of CXCL13 in a reference sample.

It may be determined that the subject is not to be treated when IgM isotype antibodies which recognise LGI1, and/or CXCL13, are absent. It may be determined that the subject is not to be treated when the level of IgM isotype antibodies which recognise LGI1, and/or the level of CXCL13, in the biological sample obtained from a subject are lower than, or the same as, the level of IgM isotype antibodies which recognise LGI1, and/or the level of CXCL13, in a reference sample.

In another aspect, there is provided a method of identifying a subject that has been diagnosed with an antibody-associated autoimmune disease, wherein the antibody recognises a specific antigen, who is likely to benefit from treatment wherein the method comprises:
a) providing a biological sample obtained from a subject; and
b) determining the presence or level of IgM isotype antibodies which recognise the specific antigen.

In another aspect, there is provided a method of identifying a subject that has been diagnosed with NMOSD who is likely to benefit from treatment wherein the method comprises:
a) providing a biological sample obtained from a subject; and
b) determining the presence or level of IgM isotype antibodies which recognise AQP4.

It may be determined that the subject is likely to benefit from treatment when IgM isotype antibodies which recognise AQP4 are present. It may be determined that the subject is likely to benefit from treatment when the level of IgM isotype antibodies which recognise AQP4 in the biological sample obtained from a subject are higher than the level of IgM isotype antibodies which recognise AQP4 in a reference sample.

It may be determined that the subject is not likely to benefit from additional treatment or that treatment may be reduced when IgM isotype antibodies which recognise AQP4 are absent. It may be determined that the subject is not likely to benefit from additional treatment or that treatment may be reduced when the level of IgM isotype antibodies which recognise AQP4 in the biological sample obtained from a subject are lower than, or the same as, the level of IgM isotype antibodies which recognise AQP4 in a reference sample.

In another aspect, there is provided a method of identifying a subject that has been diagnosed with encephalitis who is likely to benefit from treatment wherein the method comprises:
a) providing a biological sample obtained from a subject; and
b) determining the presence or level of IgM isotype antibodies which recognise LGI1 and/or the presence or level of CXCL13.

The encephalitis may be mediated by and/or diagnosed by the presence of IgG isotype antibodies which recognise LGI1.

It may be determined that the subject is likely to benefit from treatment when IgM isotype antibodies which recognise LGI1, and/or CXCL13 are present. It may be determined that the subject is likely to benefit from treatment when the level of IgM isotype antibodies which recognise LGI1, and/or the level of CXCL13 in the biological sample obtained from a subject are higher than the level of IgM isotype antibodies which recognise LGI1, and/or the level of CXCL13 in a reference sample.

It may be determined that the subject is not likely to benefit from additional treatment or that treatment may be reduced when IgM isotype antibodies which recognise LGI1, and/or CXCL13 are absent. It may be determined that the subject is not likely to benefit from additional treatment or that treatment may be reduced when the level of IgM isotype antibodies which recognise LGI1, and/or the level of CXCL13 in the biological sample obtained from a subject are lower than, or the same as, the level of IgM isotype antibodies which recognise LGI1, and/or the level of CXCL13 in a reference sample.

In another aspect, there is provided a method of treating a subject that has been diagnosed with NMOSD, wherein the method comprises:
a) providing a biological sample obtained from a subject;
b) determining the presence or level of IgM isotype antibodies which recognise AQP4; and
c) treating the subject when IgM isotype antibodies which recognise AQP4 are present, or when the level of IgM isotype antibodies which recognise AQP4 in the biological sample obtained from a subject are higher than the level of IgM isotype antibodies which recognise AQP4 in a reference sample.

In another aspect, there is provided a method of treating a subject that has been diagnosed with encephalitis, wherein the method comprises:
a) providing a biological sample obtained from a subject;
b) determining the presence or level of IgM isotype antibodies which recognise LGI1 and/or the presence or level of CXCL13; and
c) treating the subject for encephalitis when IgM isotype antibodies which recognise LGI1 and/or CXCL13 are present, or when the level of IgM isotype antibodies which recognise LGI1, and/or the level of CXCL13, in the biological sample obtained from a subject are higher than the level of IgM isotype antibodies which recognise LGI1 and/or the level of CXCL13 in a reference sample.

The encephalitis may be mediated by and/or diagnosed by the presence of IgG isotype antibodies which recognise LGI1.

The method of any aspect may further comprise the step of determining the level of, or change in the level of, one or more other Ig isotype which recognises a specific antigen present in the biological sample obtained from the subject. The one or more Ig isotype may be an IgG, IgM, IgE, IgA, and/or IgD.

The method of any aspect may further comprise the step of determining the change in predominant epitope recognised by one or more Ig isotype present in the biological sample obtained from the subject. The one or more Ig isotype may be an IgM, IgE, IgA, and/or IgD.

The method of any aspect may further comprise the step of determining the level of, or change in the level of, one or more IgG subclasses which recognise a specific antigen present in the biological sample obtained from the subject. Additionally or alternatively the method of any aspect may further comprise the step of determining if there has been a switch in the predominant IgG subclass of the antibodies that recognise a specific antigen.

In place of determining the level or presence of IgM isotype antibodies which recognise the specific antigen, the method of any aspect may alternatively comprise the step of determining the level of, or change in the level of, one or more IgG subclasses which recognise a specific antigen present in the biological sample obtained from the subject.

In place of determining the level or presence of IgM isotype antibodies which recognise the specific antigen, the method of any aspect may alternatively comprise the step of determining if there has been a switch in the predominant IgG subclass of the antibodies that recognise a specific antigen.

A change, such as an increase or decrease, in the level of a given IgG subclass which recognise a specific antigen compared to the level of that IgG subclass which recognise the specific antigen in a reference sample, or a switch in the predominant IgG subclass in the IgG antibodies that recognise a specific antigen, may indicate a higher likelihood of relapse, or that the subject is in relapse, or that the subject is to be treated, or that the subject is likely to benefit from treatment. The subject may then be treated. The treatment may be with an anti-CD20 antibody, such as Rituximab. The treatment may alternatively or additionally be with one or more of an anti-CD20 antibody, such as Rituximab, Ofatumumab or Ocrelizumab; steroids; azathioprine; mycophenolate mofetil; agents, such as mABs, which recognise IL-6R or reduce IL-6R activity; agents, such as mAbs, which recognise CD19 or reduce CD19 activity; agents which alter the function of complement pathway components (such as Eculizumab); and bortezomib; for example.

The change in the level of a given IgG subclass which recognise the specific antigen may be an about 10% or more, about 20% or more, about 30% or more, about 40% or more, about 50% or more increase or decrease in the level of that IgG subclass which recognise the specific antigen, compared to the level of that IgG subclass which recognise the specific antigen in a reference sample.

The increase in the level of a given IgG subclass which recognise the specific antigen may be an increase to at least about 50% or more, at least about 60% or more, at least about 70% or more, at least about 80% or more, at least about 90% or more of the total IgG which recognise the specific antigen in the sample.

In one example, where the level of a given IgG subclass which recognise the specific antigen does not increase, for example to at least about 50% at least or more, at least about 60% or more, at least about 70% or more, at least about 80% or more, at least about 90% or more of the total IgG which recognise the specific antigen in the sample, this may indicate a lower risk of relapse, and that the subject is not in relapse, accordingly the subject may not need to be treated, or the subject may require no additional treatment, or the treatment administered may be reduced.

The specific antigen may be AQP4. In particular if the antibody-mediated autoimmune disease is NMOSD the specific antigen may be AQP4.

The IgG subclass may be IgG1, IgG2, IgG3, or IgG4.

The antibody-mediated autoimmune disease of any aspect may be an autoimmune encephalitis associated with the presence or increase in the level of antibodies against LGI1, CASPR2 and NMDA receptors in a subject.

The invention is based on the demonstration that in an antibody-mediated autoimmune disease, the appearance or increase in the level of IgM which recognise a specific antigen which is clinically implicated in that antibody-mediated autoimmune disease, such as AQP4 in NMOSD, and/or LGI1 in encephalitis, is a robust and statistically significant indicator or predictor that a subject is in relapse or is likely to enter relapse. A strong negative predicative value can also be demonstrated by the absence of IgM which recognise the specific antigen, and/or by looking at the levels of CXCL13. The inventors also demonstrate that the predictive value of relapse or no relapse can be strengthened by measuring the relative level of different IgG subclasses in a sample; a switch in the predominant IgG subclass is a further positive indicator that a subject is in relapse or is likely to enter relapse, whilst no change in the predominant IgG subclass can strengthen the negative predictor value of relapse. The invention therefore allows the decision to be made whether to treat a subject or not, or whether to change their treatment. This can allow the reduction in severity of clinical manifestations of a relapse in a patient who is in or likely to enter relapse, whilst deciding not to treat a patient who is not in relapse and/or is unlikely to enter relapse around the time of any method disclosed herein may prevent unnecessary treatment, unnecessary side effects and unnecessary spending; for example, Rituximab treatment for a patient with NMOSD can be very expensive. Treatments for antibody-mediated autoimmune disease also have significant side effects and thus causing avoiding unnecessary treatment is important.

### General Definitions

The term “antibody-associated autoimmune disease” refers to an autoimmune disease in which the presence, or an increase in the level of, antibodies to a specific antigen, are linked to the disease. Such antibodies may or may not contribute to disease establishment or progression. That is, such antibodies may be correlative and not causative of the autoimmune disease.

The term “antibody-mediated autoimmune disease” refers to an autoimmune disease in which the presence, or an increase in the level of, antibodies to a specific antigen, contribute to disease establishment or progression and/or one or more symptoms of the disease.

The term ‘relapse’ refers to a recurrence of the clinical manifestation or manifestations associated with the antibody-mediated autoimmune disease, typically caused by an increase in production of antibodies.

The terms ‘high’ and ‘higher’ as used herein may refer to an increase of at least 10% or more, at least 20% or more, at least 30% or more, at least 40% or more, at least 50% or more, at least 100% or more, at least 2000% or more, at least 500% or more, at least 1000% or more increase in a parameter that is measured. The terms ‘high’ and ‘higher’ as used herein may alternatively refer to at least a 2-fold or more, at least a 5-fold or more, at least a 10-fold or more, at least a 20-fold or more, at least a 50-fold or more, at least a 100-fold or more, at least a 500-fold or more, at least a 1000-fold or more, at least a 10000-fold or more increase in a parameter that it measured.

The terms low′ and ‘lower’ as used herein may refer to an increase of at least 10% or more, at least 20% or more, at least 30% or more, at least 40% or more, at least 50% or more, at least 600% or more, at least 70% or more, at least 800% or more, at least 90% or more decrease in a parameter that is measured.

The term ‘biological sample’ refers to a sample of biological fluid obtained from a subject of interest. Preferred biological samples include, but are not limited to, blood, serum, plasma, saliva, lymph node aspirates such as deep cervical lymph node aspirates, and cerebrospinal fluid. In addition, the person skilled in the art would realise that some test samples would be more readily analysed following a fractionation or purification procedure, for example, separation of whole blood into serum or plasma components. Preferably, the biological sample is from blood or a lymph node aspirate, such as a deep cervical lymph node aspirate. The data presented herein suggests an underlying biological mechanism involving germinal centre reactions, and therefore lymph node aspirations may provide a more direct measure of changes in the levels of IgM and subclass of IgG. As germinal centres are also known to be sites of epitope diversification and isotype switching (also referred to as class switch recombination), it is possible the presence or increase in IgM, and IgG subclass switches, are other features of germinal centres.

The biological sample may be a plasma sample obtained from a subject.

As used herein, a ‘reference sample’ may be a corresponding sample, for example a blood sample or a lymph node aspirate such as deep cervical lymph node aspirate, obtained from the subject at a different time point, for example when the subject is determined to not be in relapse. Alternatively, the reference sample may be a corresponding sample, for example a blood sample or a lymph node aspirate such as deep cervical lymph node aspirate, obtained from a different subject who has not been diagnosed with the same antibody-mediated autoimmune disease as the first subject.

The step of obtaining a sample preferably does not form part of the invention.

The term ‘level’ as used herein refers to the amount or concentration of the IgM isotype antibodies which recognise the specific antigen, such as AQP4, and/or LGI1; or the amount or concentration of CXCL13 contained in the biological sample.

The phrase ‘disease status’ includes any manifestation of the antibody-mediated autoimmune disease. Preferably the disease status refers to the presence or likelihood of a relapse in the subject, and/or the whether the subject will benefit from treatment.

The level of IgM isotype antibodies which recognise the specific antigen, such as AQP4 and/or LGI1, and/or the level of CXCL13, may be evaluated by any suitable method. For example if protein levels are to be determined any of the group comprising immunoassays, spectrometry, western blot, ELISA, immunoprecipitation, slot or dot blot assay, isoelectric focussing, SDS-PAGE and antibody microarray immunohistological staining, radio immuno assay (RIA), fluoroimmunoassay, an immunoassay using an avidin-biotin or streptoavidin-biotin system, etc. and combinations thereof may be used. These methods are well known to persons skilled in the art. Other methods may also be used.

The methods described herein may be carried out in vitro.

The subject may be a mammal, for example, a human, a dog, cat, horse, cow, monkey, ape, rodent, hamster, rat, or guinea pig. Preferably, the subject is a human.

The person skilled in the art will appreciate that preferred features of any one embodiment and/or aspect of the invention may be applied to all other embodiments and/or aspects of the invention.

There now follows by way of example only a detailed description of the present invention with reference to the accompanying drawings, in which:

### BRIEF DESCRIPTION OF THE DRAWINGS

FIG. 1—demonstrates serological associations with relapses and rituximab (RTX) administration in patients with Neuromyelitis Optica spectrum disorders (NMOSD). A. The effect of RTX administration on relapses in 35 NMOSD patients administered RTX at time zero. Timing of lymph node (LN) and peripheral blood mononuclear cell (PBMC) sampling shown (star). B. Annualised relapse rate (ARR) at last follow up compared between patients pre-RTX and post-RTX (p<0.001), and to those not administered RTX (p<0.001, Mann-Whitney U tests). C. Aquaporin 4-Immunoglobulin G (AQP4-IgG) end-point dilutions were not reduced after several RTX infusions (p=0.99, Kruskal-Wallis test). D. Heat-maps to represent associations between relapses (x) and the dominant AQP4-IgG subclass (>50% of total AQP4-IgG, top panels), AQP4-IgM levels (middle panels) and total AQP4-IgG levels (lower panels) in patients either administered RTX (n=22) or naïve to RTX (n=28). E. Detailed examples in individual patients (6, 7 and 10) with negative values indicating days prior to the first RTX infusion.

FIG. 2—shows that cervical lymph node aspirations contain AQP4-antibodies which are abrogated after RTX administration. A. Cervical lymph nodes (LN) across anatomical levels were aspirated under ultrasound guidance. Paired blood was also sampled resulting in cellular and soluble fractions from both sites. B. Markedly different levels of total IgG (filled symbols) and IgM (empty symbols) were measured in LN aspirates (light symbol outline, IgG diluted at 1:800; IgM diluted at 1:100) versus matched sera of NMOSD patients (dark symbol outline, IgG diluted at 1:100000; IgM diluted at 1:6400). C. Differences between PBMCs and LN cell populations are highlighted by the ratio of monocytes:lymphocytes and frequencies of both transitional B cells and T-follicular helper (TfH) cells (all p<0.001, Wilcoxon signed ranks test), confirming ultrasound sampling resulted in limited contamination. D. AQP4-IgG were detected in aspirates by binding (anti-human IgG, red) to the surface of live AQP4-enhanced green fluorescent protein (EGFP) transfected HEK293T cells (green) and to the surface of live mouse astrocytes (identified with glial fibrillary acidic protein, GFAP, green). E. AQP4-IgG levels in serum (dark filled columns) and LN aspirates (light filled columns) were measured in patients naïve to RTX (n=7, of whom two went onto receive RTX: patient 5 and 9), 0.5-6 months after one RTX infusion (n=4) and 0.5-13 months after >1 RTX infusion (n=7, including two sampled longitudinally: patients 2 and 4). F. The ratio of AQP4-IgG levels (end point dilutions): total IgG levels were calculated for LNs and serum (p=0.02, Wilcoxon signed ranks test). Larger dots identify the two RTX-treated patients with detectable AQP4-IgGs, in FIG. 2E. (G) Comparisons patients who did versus did not receive RTX in absolute AQP4-IgG levels from LN aspirates (p=0.04, Mann-Whitney U test) and serum (non-significant).

FIG. 3—shows the characterisation of Aquaporin-4 specific B cells from lymph nodes and blood of patients with NMOSD. A. Single B cells (CD3⁻CD19⁺) from paired blood (left) and LN (right) samples were labelled with detection antibodies, index sorted as single cells and cultured. By day 22, ˜50% of cells proliferate and differentiate into antibody secreting cells (CD19 cells gated to show CD27, CD38 and CD138 expression). Indexing revealed the original B cell subsets: naïve, double negative (DN), IgD⁻ and IgD⁺ memory (Mem). B. AQP4-reactive IgG/Ms supernatants were identified by reactivity (red) directed against live HEK293T cells which expressed surface AQP4-EGFP (green). C. AQP4-specific B cell frequencies detected from three patients in LNs (light filled columns) and PBMCs (dark filled columns). D. The heavy and light chains of these 11 AQP4-specific B cell receptors (7 and 11 sequenced, respectively) arose from all four B cell subsets and showed varied gene families (heavy variable IGHV3 in triangle and IGHV4 in diamond) and light chain usage (kappa or lambda, in square and circle, respectively). The detected isotype in supernatants (AQP4-IgM/IgG, triangles and circles) and tissue of origin (LN or PBMCs, hollow and filled shapes) are also shown.

FIG. 4—demonstrates that intranodal B cells are rapidly and effectively depleted after rituximab administration. A. After RTX administration, B cells (CD14⁻DAPI⁻CD3⁻CD19⁺) were markedly depleted from both LN (light filled circles, p<0.001, Mann-Whitney U test) and blood (dark filled circles, p<0.001, Mann-Whitney U test). B. The depletion in LNs was more pronounced in patients after >1 versus only 1 RTX infusion (p=0.04, Mann-Whitney U test). C. From two patients sampled before and after the first RTX infusion (patients 5 and 9), and two sampled at sequential time-points after RTX infusion (patients 2 and 4), the unchanged serum AQP4-IgG levels (left) contrast with marked reductions in LN aspirates of both AQP4-IgG (middle) and LN B cell frequency (right).

FIG. 5—provides a summary of sampling and testing procedures and flow cytometry gating strategies. A. Venn diagram to show how the samples were distributed among patients with and without RTX administration and disease controls. The two patients in the middle-rightfield represent individuals for which assessments, including LN aspiration, was performed both before and after the first RTX dose. The individuals solely in the outer bottom circle represent 14 disease controls who underwent LN aspirations. B. Gating strategies. CD45 staining was available for 23 of 36 LN/PBMC sample pairs. When not included in the panel, events were accepted if positive for any of CD3, CD14, CD19, CD20, CD27 and/or CD38 (‘Boolean gate’). Representative example showing the phenotype of the CD45 positive population or events within the Boolean gate were indistinguishable. C. Overarching gating strategy for the flow cytometry data analysis. Gates boxed off with dark outline represent populations that were investigated.

FIG. 6—demonstrates that RTX administration is associated with absence of detectable AQP4-IgG/M in LN aspirates and matched sera. Both matched sera and LN aspirates from seven RTX-naïve NMOSD patients contained detectable surface astrocyte binding (and AQP4-IgG, e.g. FIG. 2D). In addition, 2/7 of these LN aspirates contained AQP4-IgM without their detection in matched sera. By contrast, astrocyte binding was only detected in 2/11 LN aspirates from patients administered RTX. A commercial antibody specific for GFAP confirmed their designation as astrocytes. Scale bar 10 μm.

FIG. 7—shows an indexed phenotype of AQP4-expressing B cells. dots of the left-hand plots represent cells found in PBMC whereas dots on the right-hand plots dots represent cells found in LN, across three patients.

FIG. 8—shows B- and T-cell subset frequencies in PBMC and LNs compared across disease controls and NMOSD patients after or naïve to RTX. A. IgD⁺CD27⁻ naïve and IgD⁻CD27⁺ memory B cell subset frequency expressed as a percentage of total CD19⁺ B-cells. B: CD20⁺CD3⁺ subset frequency (CD20⁺ T cells) expressed as a percentage of total CD3⁺ cells. C. CD19⁺CD20⁻CD38⁺ antibody-secreting cell (ASC) subset frequency expressed as a percentage of total CD19⁺ B-cells. D. CD4⁺CXCR5⁺PD-1⁺⁺ T follicular helper subset frequency expressed as a percentage of total CD3⁺ cells. For all populations, only individuals with more than 50 cells in the parent population (i.e. CD19⁺ or CD3⁺) were included. A median of 6704 B cells events (range 61-99629) was analysed. Statistical analyses with Mann-Whitney U-test after Benjamini-Hochberg correction for the 20 overall comparisons.

FIG. 9—outlines clinical characteristics of patients undergoing cervical lymph node fine needle aspirations (FNA). FNAs were performed in nine NMOSD patients treated with RTX, and five NMOSD patients naïve to RTX (overall, sampled at a median of 59 months into their disease; range 21-180). Also, FNAs were performed in 14 patients with other autoimmune and non-autoimmune neurological conditions. FNA was performed before and after RTX in two patients (NMOSD #5, NMOSD #9) and at two time-points after RTX in two other patients (NMOSD #2, NMOSD #4). From these four patients, FNA were from the same lymph node. Overall, cervical lymph node anatomical levels I, II, III and V were sampled. ARR=annualised relapse rate; AZA=azathioprine; CyP=cyclophosphamide; FNA=fine needle aspiration; GAD-Ab-E=glutamic acid decarboxylase antibody encephalitis; GlyR-Ab-E=glycine receptor antibody encephalitis; LGI1-Ab-E=leucine rich glioma inactivated 1 antibody encephalitis; MMF=mycophenolate mofetil; MTX=mitoxantrone; NMDAR-Ab-E=N-methyl-D-aspartate receptor antibody encephalitis; NMOSD=Neuromyelitis Optica spectrum disorders; Pred=prednisone; RTX=Rituximab;

FIG. 10—demonstrates the characteristics of AQP4-specific B cell receptor sequences retrieved from single B cell cultures. The table shows the patient number, tissue from which the B cell subset (naïve [CD27⁻IgD⁺]; double negative [CD27⁻IgD⁻]; IgD⁺ memory [CD27⁺IgD⁺]; IgD⁻ memory [CD27⁺IgD⁻]) was isolated and characteristics of both the heavy chains (isotype detected in culture, predicted V and J gene alleles and total number of variable region mutations; www.imgt.com) and light chains (lambda or kappa; predicted V and J gene alleles and total number of variable region mutations; www.imgt.com). From four B cells, heavy chains were not amplified (N/A).

FIG. 11—is a receiver operator characteristic curve showing sensitivity and (1-specificity) by different cut-offs of AQP4 IgM titres. Both the log titre, presence of and change in AQP4-IgM level were strongly predictive of relapse (AUC 0.67).

FIG. 12—demonstrates the results of ROC curves for different measures of IgG subclass data. IgG subclasses (either percentage or log ABC) were less predictive than IgM alone.

FIG. 13—demonstrates the proportion of relapse samples by the summed percentage difference in AQP4-IgG in a sliding window plot (30 samples wide). The sum of % change over all IgG subclasses as a surrogate for class switching. This shows a predictive value when excluding samples with an AQP4 IgM rise (AUC=0.57). This shows that the magnitude of IgG subclass switching is associated with relapse risk but only in samples without an increase in AQP4-IgM. This suggests that changes in AQP4-IgG subclass switching can act as a risk factor for relapse, however the most powerful predictor is a combination of both IgM level increase and IgG subclass switching. Given a proportion of samples are associated with relapse in the absence of either an AQP4-IgM or AQP4-IgG subclass switch, the data suggest that other unmeasured variables may be present—e.g. IgA class switching or epitope changes in the absence of detectable IgM/IgG subclass differences.

FIG. 14—is a ROC plot of the score produced by the general linear model of relapse probability ˜log 2 (AQP4-IgM difference)+AQP4-IgG subclass difference to show the sensitivity and (1-specificity). The inset shows a sliding window plot of this score against probability of relapses. A combination score based on general linear model (outset) weightings of AQP4 IgM and IgG subclass % changes showed good predictive strength (AUC=0.73). At the optimum threshold (0.199), the sensitivity is 0.78, and specificity 0.69.

FIG. 15—shows a plot of positive predictive value and negative predictive value against general linear model score, for a combination of AQP4 IgM and IgG subclass % change, time windowed and taking account between sequential samples from the subject. A very strong negative predictive value of 0.94 is demonstrated. PPV=positive predictor value, bottom line. NPV=negative predictor value, top line.

FIG. 16—shows a ROC curve showing the score from a general linear model of relapse probability˜log 2 (AQP4-IgM)+AQP4-IgG1%. Compared with FIG. 15, which does not take account between sequential samples from the subject, treating each sample as an independent data-point. The inset shows a sliding window plot of this score against probability of relapses. AQP4 IgM and AQP4-IgG1% of total AQP4-IgG were most significant associations in generalised linear model (AUC=0.71). At optimum threshold (−0.435), the sensitivity is 0.70, the specificity is 0.72.

FIG. 17—is a plot of positive predictive value and negative predictive value against general linear model score, which does not take account between sequential samples from the subject, treating each sample as an independent data-point. A positive predictive value at a threshold of >1.23 (positive predictive value=0.81, negative predictive value=0.83). At the optimum threshold from ROC curve−positive predictive value=0.37, negative predictive value=0.91. PPV=positive predictor value, bottom line. NPV=negative predictor value, top lime.

FIG. 18—Detection of AQP4-IgG subclasses. Flow cytometry gating and representative examples of AQP4-IgG1-4 subclasses detection in four NMOSD patients. AQP4=aquaporin 4; IgG=Immunoglobulin G; NMOSD=Neuromyelitis Optica spectrum disorders.

FIG. 19—Detection of AQP4-IgM by cell based assay. After depletion of IgG, AQP4-IgM were detected in serum by binding (anti-human IgM, red) to the surface of live AQP4− EGFP transfected HEK293T cells (green). AQP4=aquaporin 4; EGFP=enhanced green fluorescent protein; HEK=Human embryonic kidney; IgM=Immunoglobulin M.

FIG. 20—Demonstrates that CXCL13 levels were significantly higher in sera from relapsing versus non-relapsing LGI1-autoantibody patients and relapsing patients more frequently showed CXCL13 levels above the cut-off (70% of sera versus 30% of sera from non-relapsing patients; p<0.0001). CXCL13 was measured from (a subset of patients/samples) 142 serum samples of 48 LGI1-antibody patients. From 37 healthy control samples, a cut-off of mean plus 3 standard deviations was derived for CXCL13 levels (145 pg/ml). Based on this, from a larger cohort, 59/87 (68%) of positive CXCL13 samples were from relapsing patients and 59/84 (70%) of samples from relapsing patients were CXCL13 positive (both p<0.0001; Fisher's exact test; FIG. 20).

FIG. 21—Shows a Representative example of a patient with refractory LGI1-antibody encephalitis with persistently raised CXCL13 levels, even after LGI1-autoantibodies become very low or undetectable. This was observed in several patients with refractory disease. AEDs=anti-epileptic drugs.

### METHODS AND MATERIALS

### Participants

Clinical information was collected retrospectively from the case-notes of 63 patients with NMOSD and serum AQP4-IgG,7 selected to have available longitudinal serum samples archived at −80° C. Details included demographics, clinical features, timings of medication administration and relapse dates, from which an annualised relapse rate (ARR) was calculated (Table 1 and 2). 35/63 had been administered intravenous RTX: initially 1 g twice separated by a fortnight at onset, followed by 1 g maintenance interval doses based on return of detectable circulating CD19+ counts. Written informed consent was obtained from all participants (ethical approvals: REC16/YH/0013, REC16/SC/0224 and REC14/SC/005) and 14 with other neurological conditions (autoimmune encephalitis (n=11) and migraine (n=3)).

### Fine Needle Aspiration (FNA) Procedure

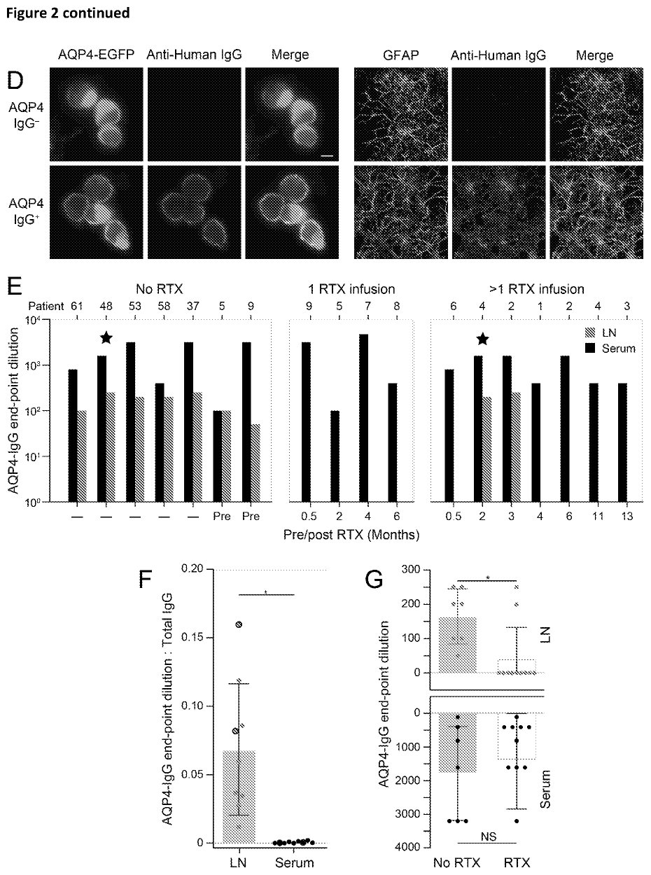

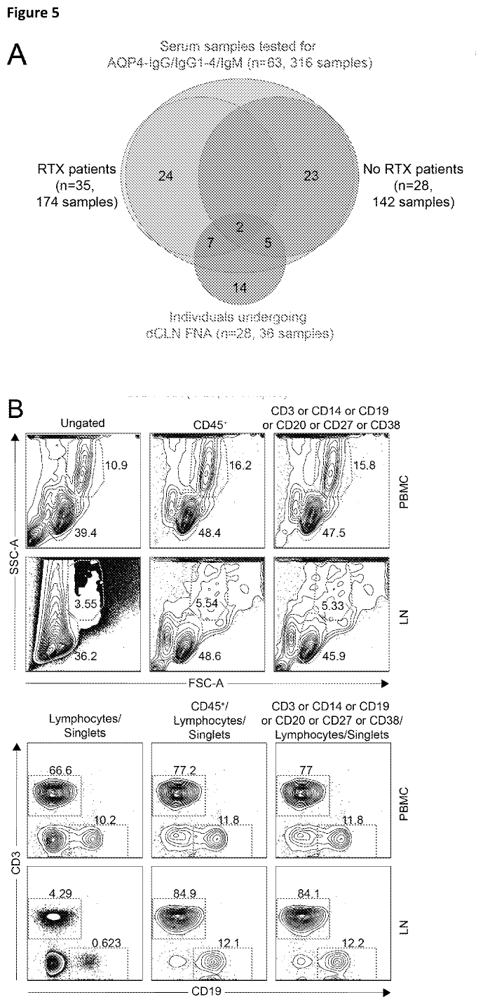

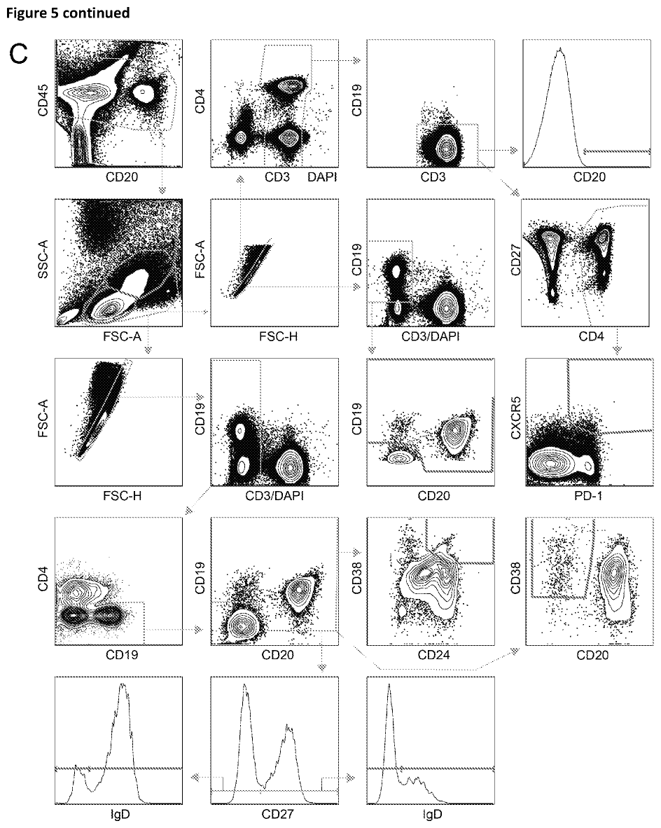

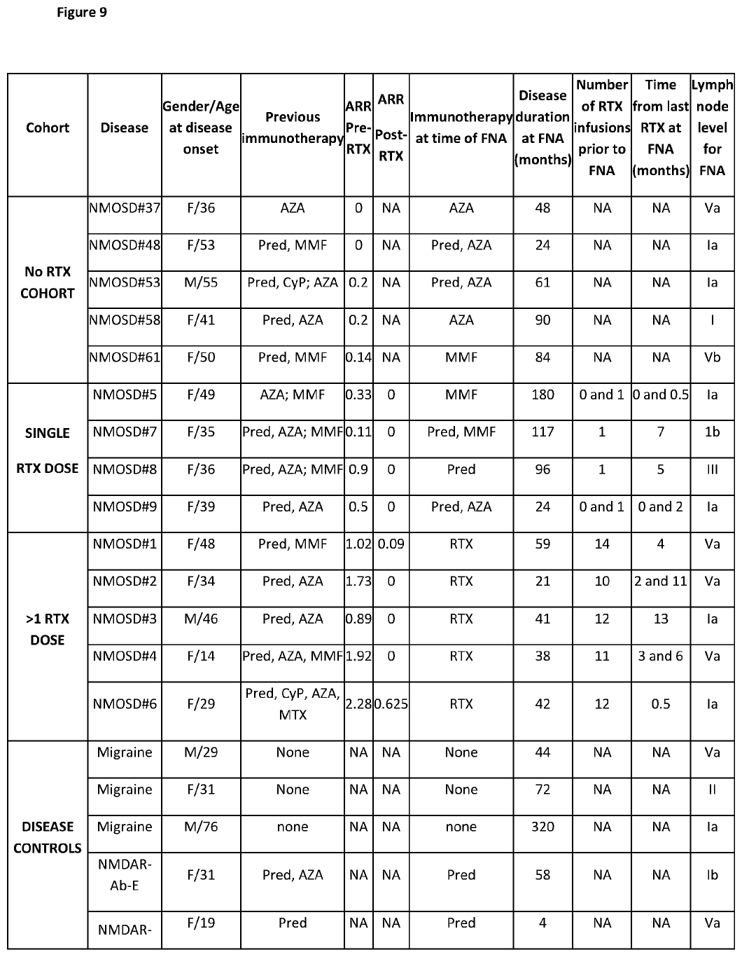

Ultrasound was used to locate dCLNs (deep cervical lymph nodes; at levels I, II III or V, FIGS. 2 and 9) which were accessed under visualisation with a 23 G needle. After two passes, aspirates were diluted in PBS and centrifuged to separate cellular and soluble fractions. The soluble fraction was tested for total IgG/M and AQP4-IgG/M (diluted 1:5), as below, and positive results confirmed with undiluted samples. The median number of live cells recovered per FNA was 1.5×10⁶ (range: 2.4×10⁵-3.5×10⁶). The overall breakdown of serum and dCLN assays is shown in FIG. 5A.

### Antibody Detection Methods

AQP4. From 63 patients, a total of 406 serum samples were tested for AQP4-IgGs and AQP4-IgMs using a well-validated immunofluorescence-based live cell-based assay.27 Briefly, HEK293T cells were transfected to express surface AQP4 (M23 isoform, C-terminally fused to enhanced green fluorescent protein; EGFP) and incubated with patient serum (starting dilution 1:20) or lymph node aspirates (starting dilution 1:50) prior to fixation and washing. Subsequently, Alexafluor-conjugated secondary antibodies targeting Fc regions of IgM (A21216; Invitrogen) or IgG (709-585-098; Jackson Labs) permitted detection by visualisation. All samples were titrated to end-point dilutions. Prior to AQP4-IgM determination, protein G sepharose beads (17-0618-01; GE, UK) were used to deplete IgG and prevent the IgG-IgM cross-competition likely in unfractionated sera.

To quantify AQP4-IgG subclasses, all 316/406 sera with an end-point dilution >1:20 (1:50-1:40000), from 50/63 patients, were used to label transiently transfected AQP4-EGFP expressing HEK293T cells in suspension and, after washing and fixation, bound IgGs were detected with subclass specific antibodies (IgG1 Hinge-AF647 (9052-31), IgG2 Fc-AF 647 (9070-31), IgG3 Hinge-AF 647 (9210-31), IgG4 Fc-PE (9200-09), Southern Biotech). Subsequently, DAPI was added prior to analysis with an Attune NxT flow cytometer. As described previously for other autoantibodies,28 AQP4-IgG subclass levels were calculated by the delta median fluorescence intensity of the transfected (single cells/viable/GFP positive gates) minus untransfected (single cells/viable/GFP negative gates) cells and normalized antibody binding capacities were calculated with calibration beads (Quantum Simply Cellular microspheres; Bangs Laboratories). The cut-off was determined for each subclass using 10 healthy control serum samples (mean value plus three standard deviations, SD).

Total IgG and total IgM were measured by ELISA (Bethyl Laboratories).

LGI1. LGI1-IgG and LGI1-IgM antibodies were sought by live cell-based assays, as previously described (Irani et al., 2010, Brain, 133:2734-2748) in 420 IgG-depleted serum samples from 112 LGI1-autoantibody patients (20 relapsing), 60 healthy controls, and 30 NMDAR-autoantibody patients. IgG depletion was undertaken to prevent likely higher affinity IgG inhibition of their binding.

Primary Rat Cultures of Astrocytes and Neurons

Mixed neuronal-astrocyte cultures were prepared from rat hippocampi at embryonic day 18, as described previously.29 Briefly, hippocampi were digested in trypsin, mechanically dissociated and plated with neurobasal medium/B27 supplement (1:50, Thermo Fisher) without anti-proliferate additives. After 21-28 days in vitro, patient sera (1:100) or dCLN supernatants (1:50), both diluted in conditioned media, were incubated with the live cells for 30 min at 37° C. and fixed with 4% PFA. To visualise bound human antibodies, either a goat anti-human IgG or IgM Fc cross absorbed unconjugated secondary antibody was applied (Thermo Fisher; 1:750), followed by a fluorescently conjugated tertiary antibody (donkey anti-goat IgG, AlexaFluor-568; A-11057, Thermo Fisher). To identify astrocytes, cells were permeabilized (0.1% Triton-X-100) and incubated with a commercial antibody which recognise glial fibrillary acidic protein (GFAP; DAKO, Z0334; 1:2000) and with a detection goat anti-rabbit IgG secondary (AF488, A-11008, Thermo Fisher; 1:750).

Flow Cytometry and Cell Sorting

Fresh LN mononuclear cells and matched peripheral blood mononuclear cells (PBMCs) were isolated on a Ficoll density gradient. Cells were incubated with normal mouse serum to block nonspecific binding, and surface phenotypes determined with commercial fluorochrome-conjugated antibodies: CD3 (UCHT1, Pacific Blue, BioLegend), CD14 (HCD14, Pacific Blue, BioLegend), CD45 (HI30, AF700, BioLegend), CD19 (SJ25C1, APC-Cy7, BD Biosciences), CD20 (2H7, BV711, BioLegend), CD24 (MLS, BV510, BioLegend), CD27 (O323,BV605, BioLegend), IgD (IA6-2, FITC, BD Biosciences), CD38 (HIT2, PE, BD Biosciences), CD4 (RPA-T4, PE-CF594, BD Biosciences), CXCR5 (J252D4, PE-Cy7, BioLegend) and PD-1 (RMP1-30, APC, BioLegend). Subsequently, cells were washed and DAPI was added prior to analysis with an Attune NxT flow cytometer. Flow cytometric data were manually gated using FlowJo software (Treestar Inc.; FIG. 5B-C).

Single B Cell Cultures and Immunoglobulin Chain Retrieval

A FACS Aria III was used to index-sort single B cells, prelabelled with the above antibodies which recognise CD19, IgD and CD27, into individual wells of 96-well plates where they were cultured with MS40L-low feeder cells in RPMI1640 (Thermo scientific, kind gift from Dr G Kelsoe) containing 10% FBS (Thermo scientific), 13-Mercaptoethanol, penicillin-streptomycin (10,000 U/mL), Thermo scientific), HEPES (1 M, Thermo scientific), sodium pyruvate (100 mM, Thermo scientific), MEM non-essential amino acids (100×; Thermo scientific) and Glutamax (100×, Thermo scientific). Cultures were supplemented with recombinant human IL-2 (100 μg/mL), IL-4 (100 μg/mL), BAFF (100 μg/mL) and IL-21 (50 μg/mL, all Peprotech) and maintained at 37° C. in 5% CO2 with half the media replaced twice weekly. On day 22, culture supernatants were harvested for AQP4-IgG and -IgM detection. FloJo software linked the cells corresponding to positive wells with their surface phenotype. From these wells, transcripts were preserved (x) and heavy and light chain PCRs were performed, as previously described.30 Finally, AQP4-specific variable region sequences were analysed using www.ncbi.nlm.nih.gov/igblast and www.imgt.org.

Statistical Analysis

GraphPad Prism (v8; GraphPad Software Inc, La Jolla, CA), R (R Core Team R: A language and environment for statistical computing, 2017) and Adobe Illustrator were used for statistical analysis and data presentation.

### EXAMPLES

### Example 1-AQP4-IgG Subclass and AQP4-IgM Dynamics Associate with Clinical Relapses

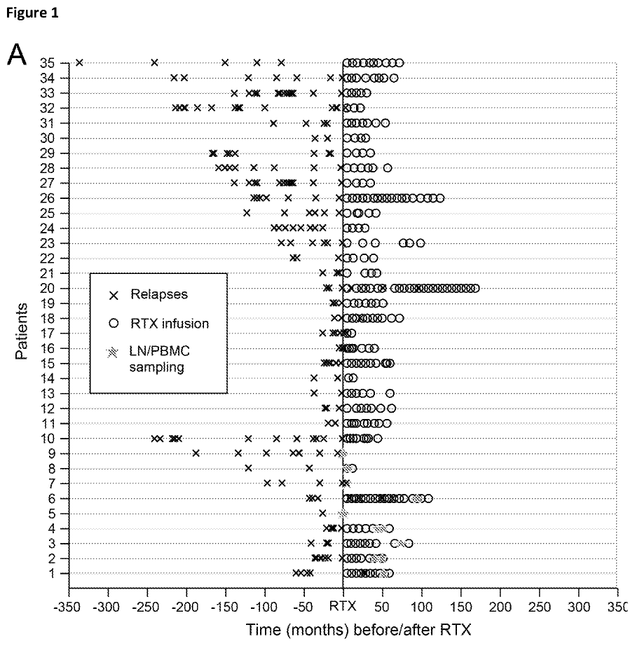

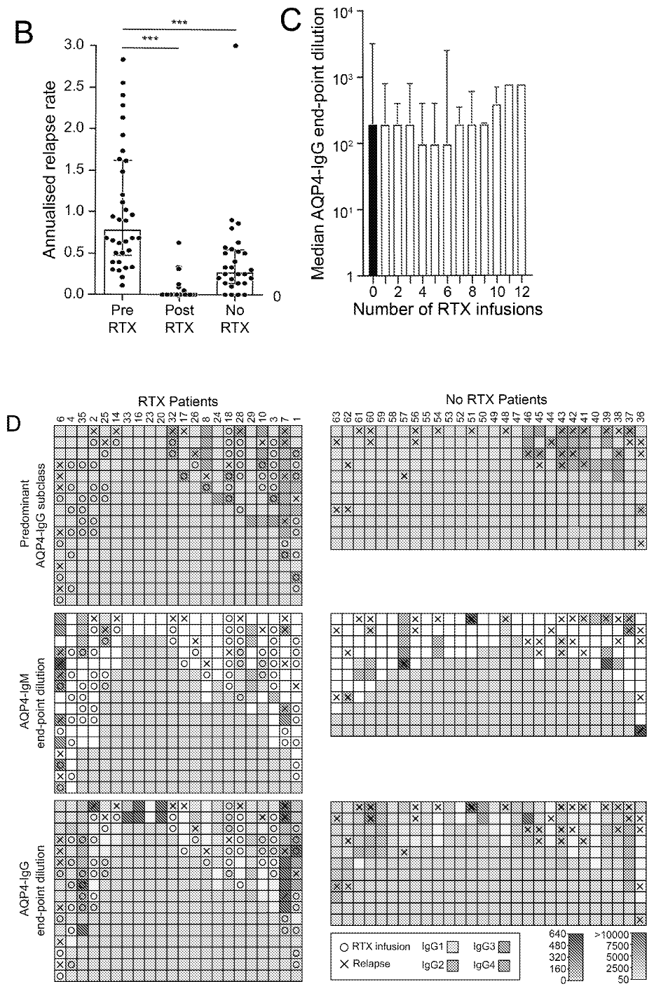

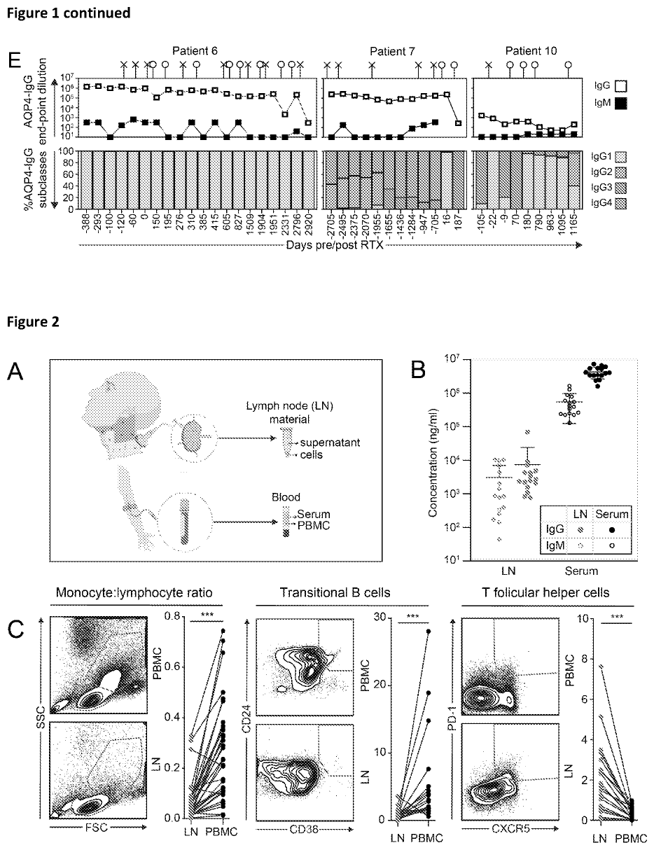

RTX administration in 35 of 63 NMOSD patients (median of 7 infusions per patient, range 1-14) was associated both with a significant reduction in the ARR (p<0.001; Mann Whitney U test, FIG. 1A-B) and the successful discontinuation of other immunotherapies in 31/35 (89%).9, 31 Also, as expected, longitudinal RTX infusions (median follow-up 50 months, range 20-135) were not associated with significant reductions in the median serum AQP4-IgG levels (FIG. 1C).

To determine whether peripheral blood could offer insights into this clinical-serological dissociation, AQP4-IgG subclasses and AQP4-IgM levels were measured from the longitudinal samples available in 50 patients (FIG. 1D-E). IgM has a half-life of five days and shifts in IgG subclasses are suggestive of active class switch recombination: hence, both represent surrogates of recent GC (germinal centre) activity. 10/22 (46%) RTX-treated patients and 17/28 (61%) who did not receive RTX exclusively showed the recognised IgG1 predominance of AQP4-IgG throughout their disease course. Therefore, overall, IgG3>2>4 was the dominant serum AQP4-IgG subclass from at least one time point in 23/50 (46%) patients. A switch in the dominant AQP4-IgG subclass was noted around the time of 24/61 (39%) clinical relapses but only at 40/255 (16%) timepoints remote from relapses (p=0.0001, Fisher's exact test). If this shift in AQP4-IgG subclasses around relapses represented naïve B cells initiating de novo GC reactions, one prediction might be that AQP4-IgMs should also be generated. Indeed, across multiple time points, serum AQP4-IgM were detected in 22/50 (44%) patients, preferentially in association with 29/61 (48%) relapses versus only 37/316 (12%) samples taken remote from relapses (p<0.0001, Fisher's exact test). By contrast to relapses, rituximab administration was not significantly associated with either AQP4-IgM or a shift in the dominant AQP4-IgG subclass (data not shown). Overall, the presence of either AQP4-IgM or an altered dominant AQP4-IgG subclass were closely associated with relapses (43/61 vs 73/255; odds ratio 6.0 (range 3.3-10.8); p<0.0001).

Similar observations remained robust in individuals. For example, in three individual patients, a switch of the predominant subclass was observed around some relapses (patients 7 and 10), and recurrent AQP4-IgM spikes in all, were often associated with relapses in patient 6. Taken together, these findings implicate GC activity as a source of AQP4-antibody production around the time of relapses.

### Example 2—Local Synthesis of AQP4-IgG, Observed in dCLN Aspirates from Patients with NMOSD, is Abrogated by RTX

To directly test the hypothesis in patients, 36 dCLN aspirations were performed in 14 patients with NMOSD (n=18 aspirations) and in 14 disease controls (n=18 aspirations) (FIG. 2 and FIG. 9). A comparison of sera versus dCLN aspirates within individuals showed ˜1500- and ˜500-fold lower total IgG and IgM levels, respectively (FIG. 2B). Further, minimal blood contamination was confirmed by the markedly different lymphocyte populations observed in dCLNs and PBMCs (FIG. 2C): dCLNs showed lower proportions of monocytes (p<0.0001) and transitional B cells (CD19⁺CD20⁺CD24⁺CD38⁺; p<0.0001) but higher frequencies of T follicular helper cells (Tfh; CD3⁺CD4⁺PD-1⁺CXCR5⁺, p<0.0001, all Wilcoxon signed ranks test).(26)

dCLN aspirates from RTX-naïve NMOSD patients showed AQP4-IgG in 7/7 samples (100%), detected by IgG binding to the extracellular domain of HEK293T-expressed AQP4 and to the surface of live astrocytes (FIG. 2D). In these seven patients, serum AQP4-IgGs ranged from end point dilutions of 1:100-1:3200 (median 1:400) and the ratio of AQP4-IgG:total IgG levels was significantly higher in dCLN aspirations versus matched sera (p=0.02; Wilcoxon signed ranks test, FIG. 2D-G). These findings are indicative of local (‘intranodal’) AQP4-IgG synthesis. This concept was further supported by the observation of AQP4-IgMs in two dCLN aspirates without their detection in matched sera (FIG. 2D and FIG. 6). By contrast to these findings, after RTX administration AQP4-IgGs were detected from only 2/11 (18%) dCLN aspirates sampled over several months (FIG. 2E-G; p=0.002, Fisher's exact test). These collective observations show that intranodal synthesis of AQP4-IgG is effectively abrogated by RTX. Hence, the deletion of AQP4-specific B cells in dCLNs by RTX may represent one mechanism to explain its clinical efficacy without an alteration in serum AQP4-IgG levels.

### Example 3—AQP4-Specific B Cells in LNs and Blood

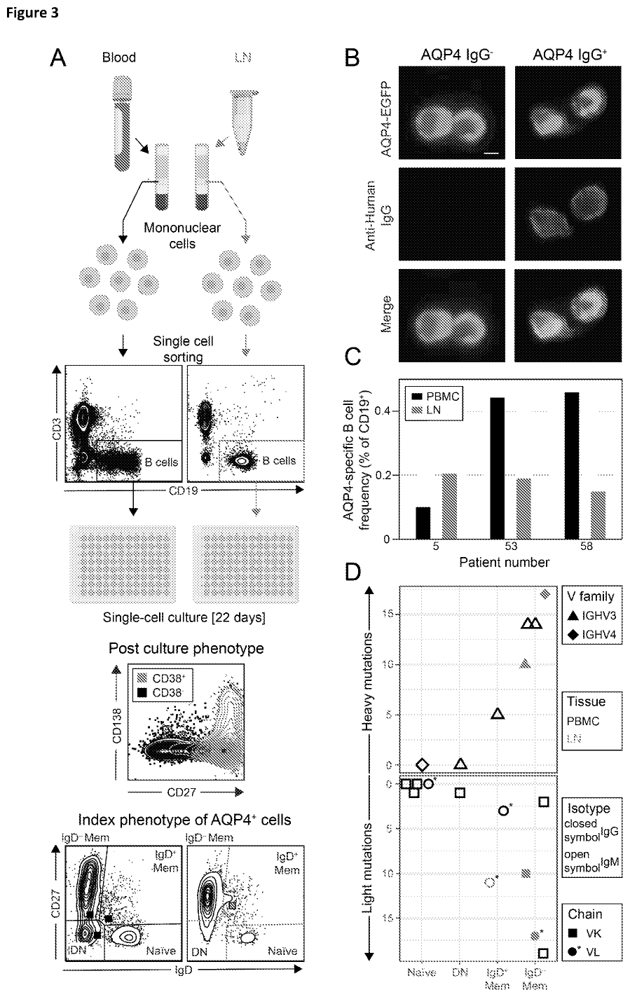

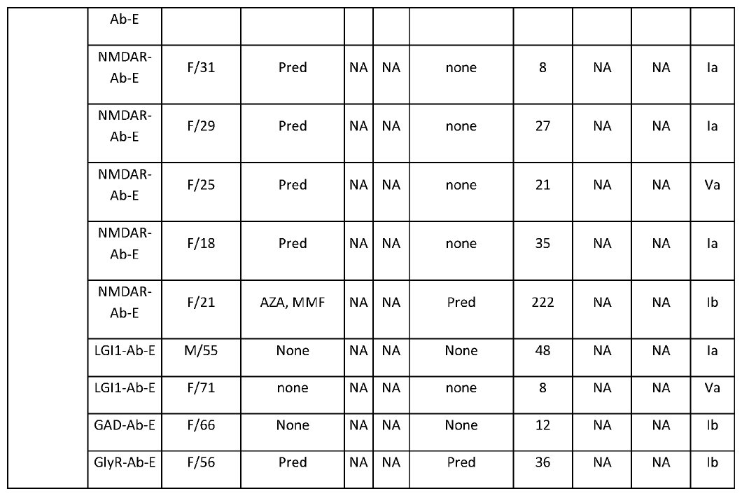

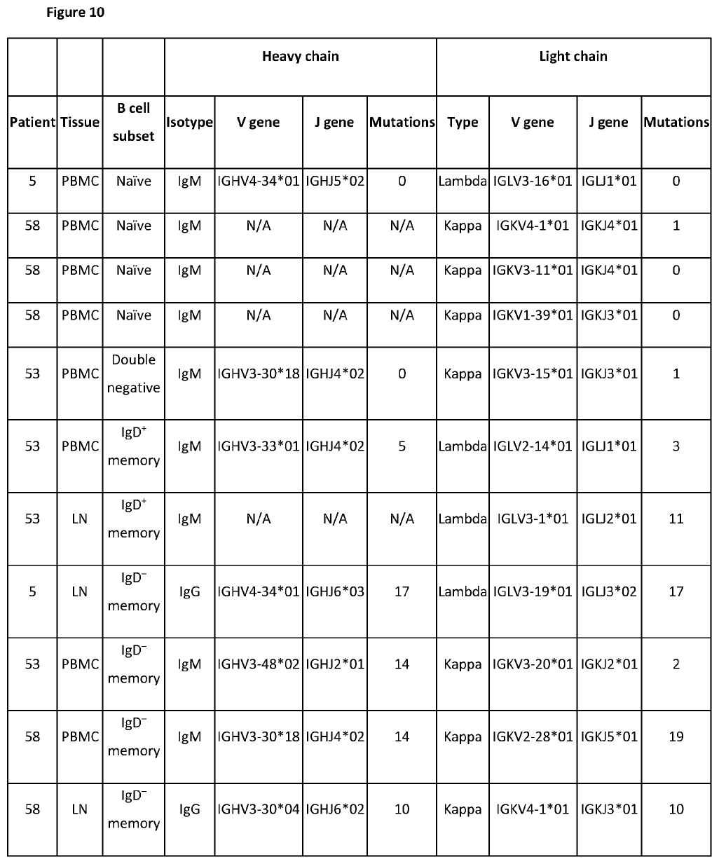

To identify AQP4-specificity within B cell subsets, single B cells from both dCLN and circulation were index sorted and individually exposed to cytokines which induced proliferation and differentiation into multiple clonally-related antibody secreting cells (CD19+CD27++CD38++, and often, CD138+; FIG. 3A). After these 22 days, the culture supernatants were assessed for AQP4-reactivities. From three patients, 11 of 8293 cultured B cells secreted either AQP4-IgM or G (FIG. 3B). Upon accounting for the ˜50% ‘catch rate’ of these cultures (data not shown), ˜0.2% of CD19+ B cells from blood and LNs showed AQP4-specificity (FIG. 3C). Cell indexing revealed that the AQP4-specific B cells derived from naïve (n=4), double negative (n=1), IgD+ memory (n=2) and IgD− memory (n=4) populations (FIG. 3A and D; FIG. 7). All three dCLN AQP4-antibodies arose from within memory subsets and contained the only two AQP4-reactivities detected as IgGs in supernatants. Cognate paired heavy and light chains from naïve B cells were unmutated with increasing mutation loads observed through double negative, IgD+ memory and IgD− memory cells. All showed highly variable V(D)J allele usage and expressed ˜2:1 kappa:lambda chains (FIG. 3D and FIG. 10). In summary, AQP4-specific B cells were detected in both PBMCs and dCLNs from patients with NMOSD, within genetically diverse lineages which arose from multiple early and late B cell subsets.

### Example 4—Effective Depletion of B Cells from dCLNs after RTX

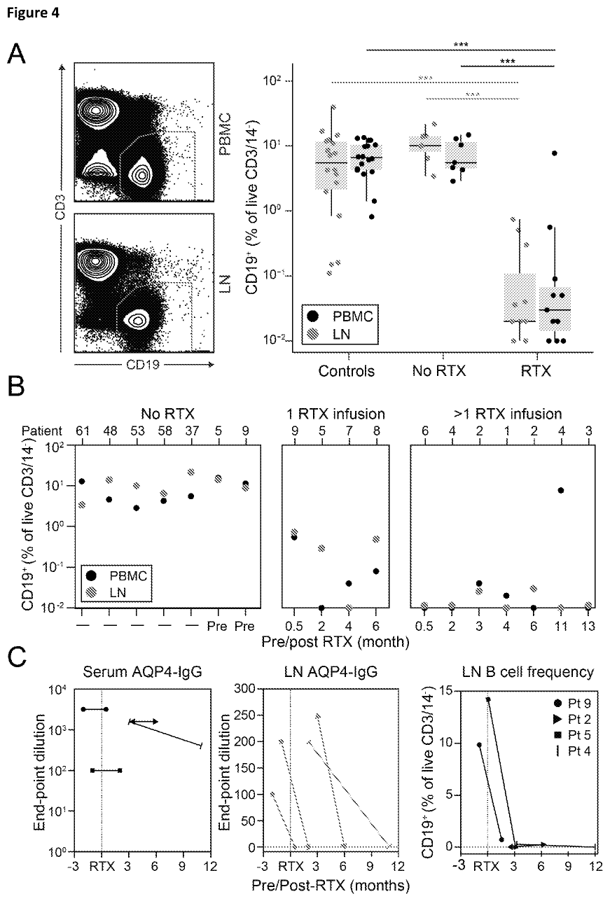

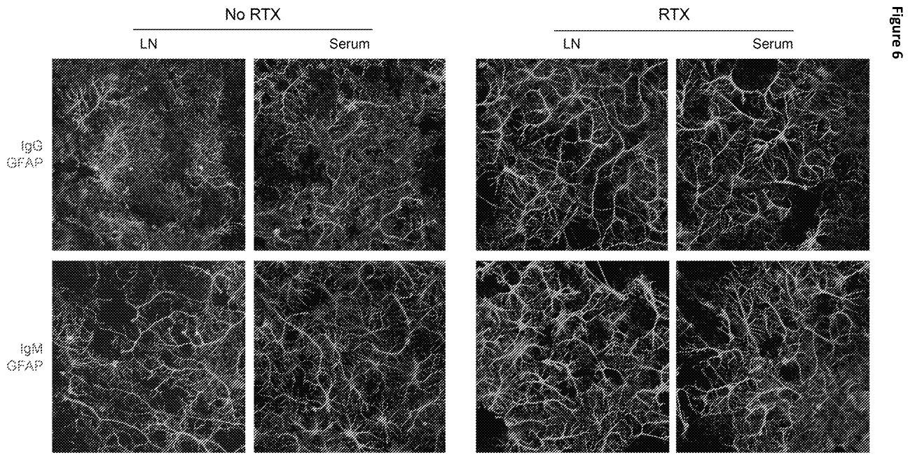

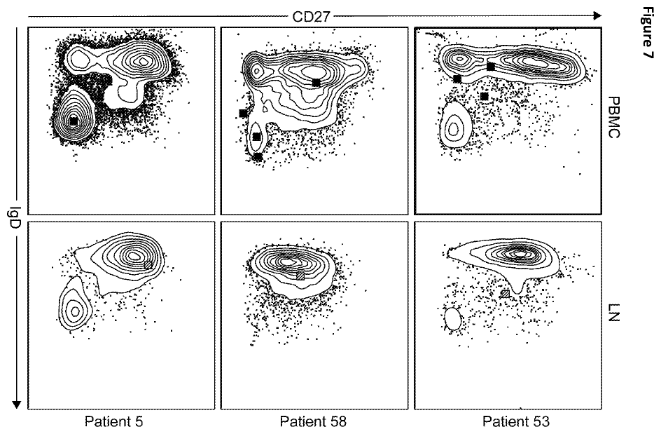

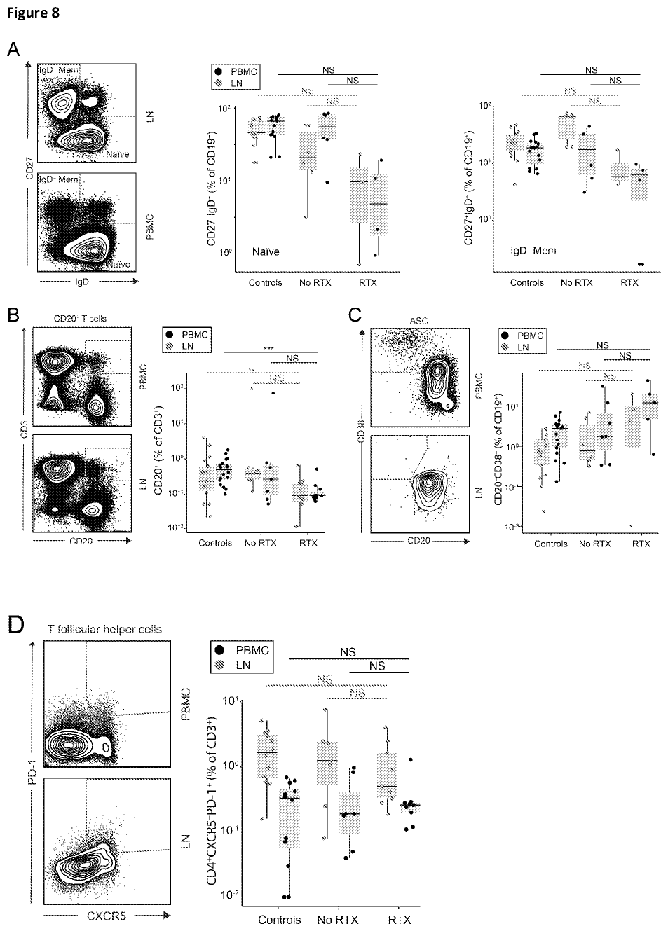

RTX administration was closely associated with a pronounced loss of CD19+ B cells observed from both PBMCs and dCLNs (p<0.001, Mann Whitney U test, FIG. 4A). B-cell subset composition was unchanged (FIG. 8A). By contrast to disease controls, there was a modest reduction in CD20+ T cell frequencies from both PBMCs and dCLNs (FIG. 8B) without significant alterations of antibody secreting cell or TfH frequencies (FIG. 8C-D). The intranodal B cell depletion was most marked in the NMOSD patients who had received >1 RTX infusion (mean of 14 dCLN B cells, range 0-56) versus those who had received a single infusion (mean of 84 dCLN B cells, range 9-230; p=0.04, Mann Whitney U test) or those who were naïve to RTX (mean of 18817 dCLN B cells, range 1602-65276; p<0.0001, Mann Whitney U test). The intranodal B cell depletion persisted for several months from RTX dosing, despite some early repopulation of circulating B cells (FIG. 4B). To confirm these results longitudinally within individuals, AQP4-IgG and B cell data were analysed from four patients who underwent paired sampling of blood and the same dCLN on two occasions, both before and after RTX administration (n=2) and twice following RTX (n=2, FIG. 4D). In all cases, the serum AQP4-IgG levels were unchanged whereas AQP4-IgG levels in dCLNs fell from end point dilutions of 1:100-1:250 to undetectable, and the dCLN B cell populations became (n=2) or remained undetectable (n=2).

### Example 5—Combinatory Measurement of IgM and IgG Subclass Changes Provides Strong Predictive Power of Relapses

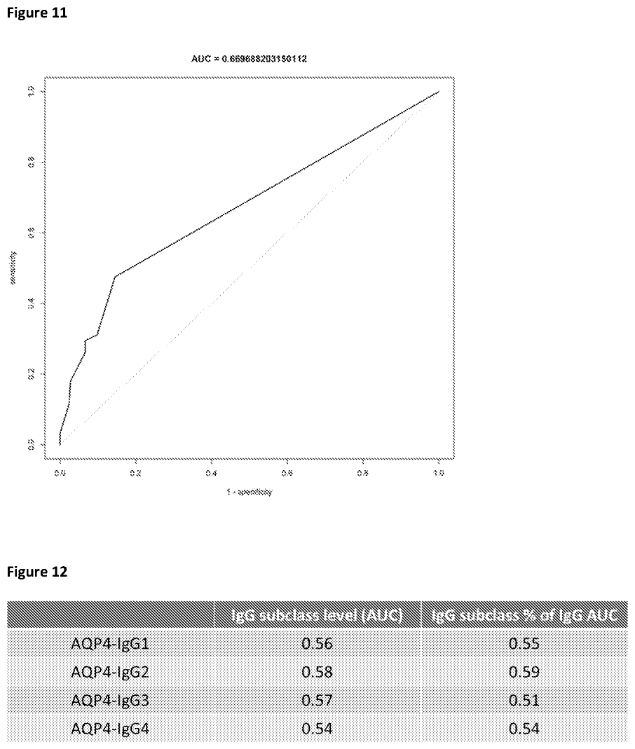

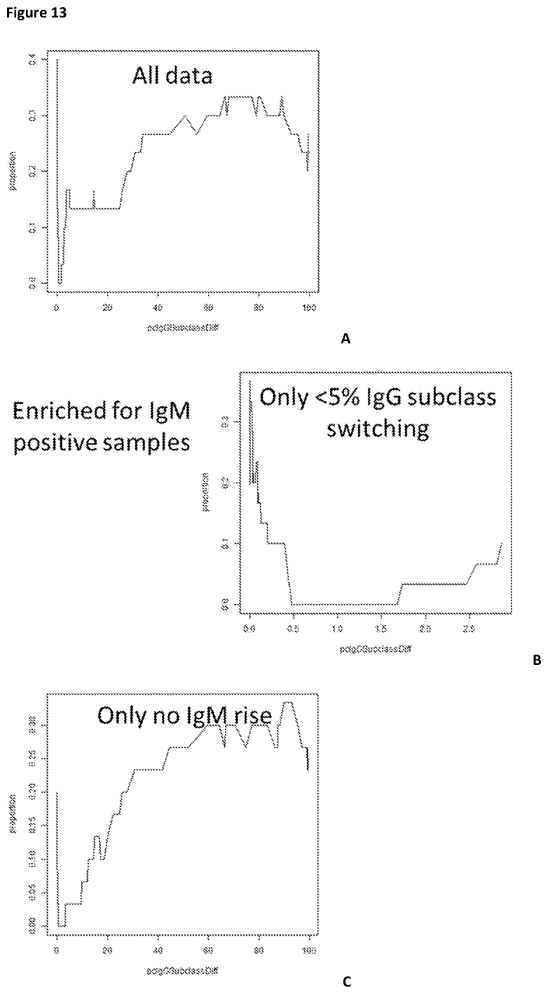

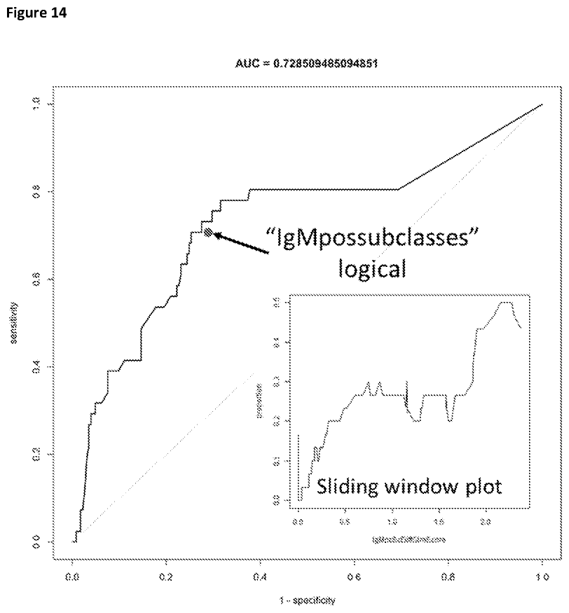

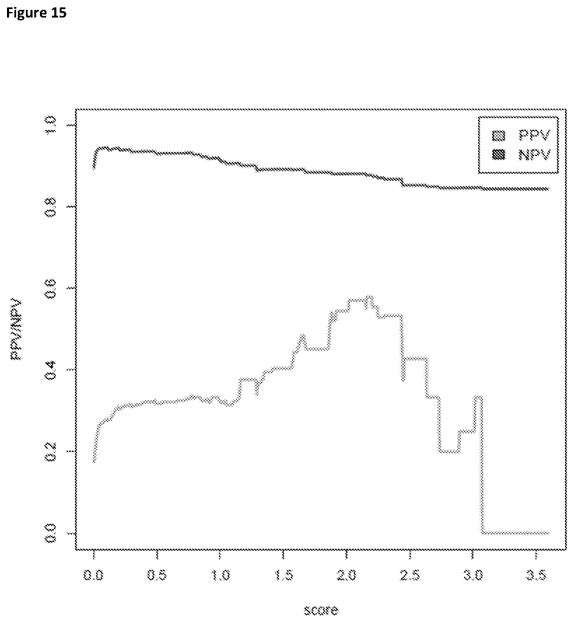

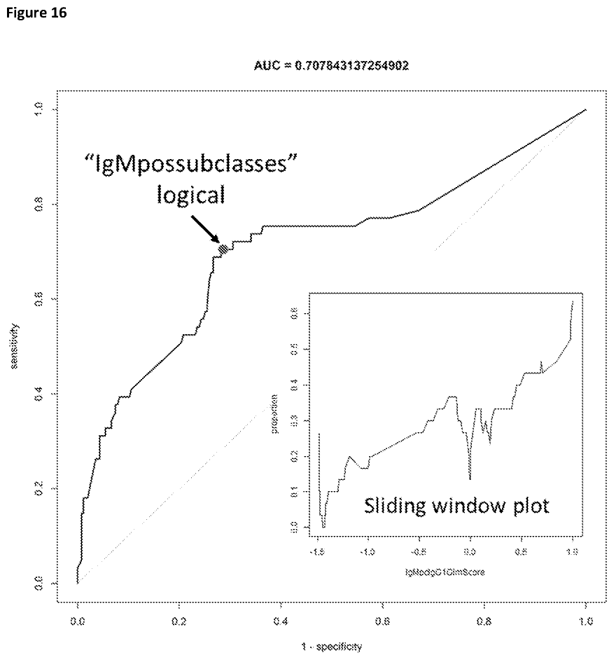

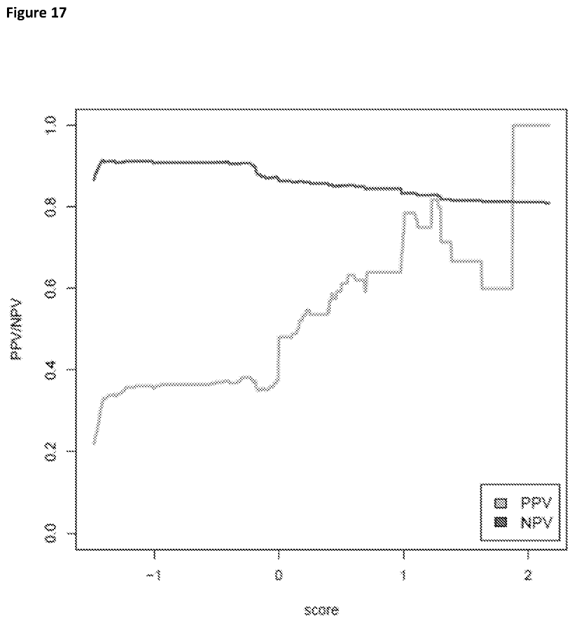

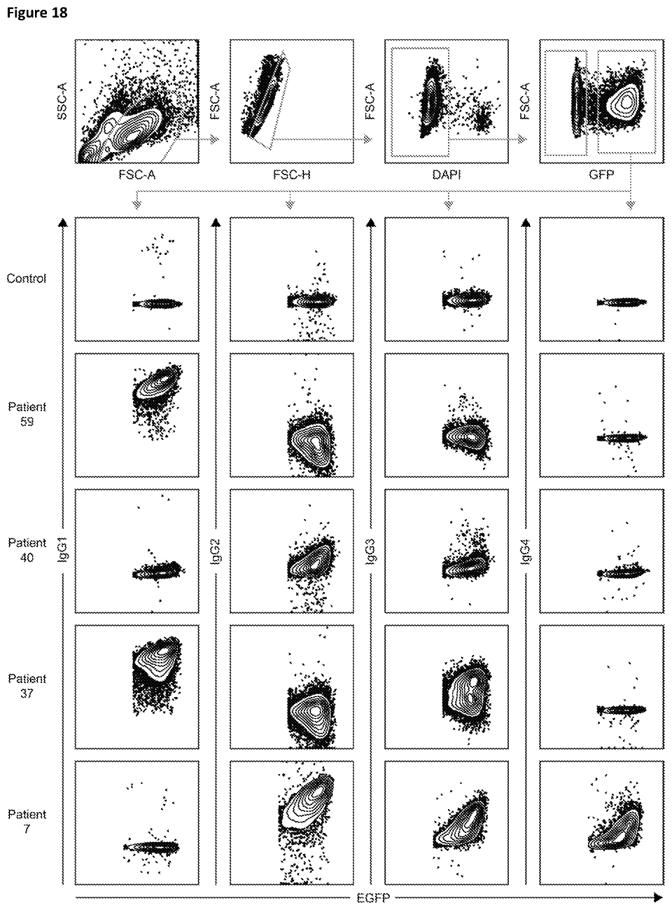

FIGS. 11-17 demonstrate that a combination of IgM and IgG subclass % or IgG switching provide the strongest predictive power for relapses. One advantage of a continuous score over a binary score is that the threshold can be adjusted to maximise sensitivity, specificity, positive predictive value or negative predictive value as required by the clinical application. These parameters can reach a negative predictive power of 0.94 and positive predictive value of 0.81. Hence, these represent effective tools for clinical decision making.

### Example 6—LGI1-IgM is Indicative of Relapse

The data demonstrates that LGI1-IgM antibodies show similar clinical associations to AQP4-IgM antibodies. By contrast to patients with AQP4-antibodies, patients with LGI1-antibodies are typically elderly males with a median age of onset around 65 years of age and a 2:1 male:female ratio. Further, around 10-40% of patients reported in the literature show relapses. Hence, the utility of autoantigen-specific IgMs is important to a broader demographic and to disease with frequent relapses.

From the first sample available from each patient, LGI1-IgG antibodies were detected, as expected. After IgG depletion using protein G beads, these LGI1-autoantibody patient sera had absence of detectable LGI1-IgG antibodies, confirming complete depletion. No healthy control or NMDAR-autoantibody patients had detectable LGI1-IgM antibodies. 16/420 samples, from 8/112 patients, showed detectable LGI1-IgM antibodies with end-titres ranging from 1:20-1:160.

| | LGI1-IgM positive | LGI1-IgM negative |
|---|---|---|
| | (n = 8) | (n = 104) |
| Relapsing (n = 9) | 7 | 2 |
| Non-Relapsing (n = 103) | 1 | 102 |

7/8 patients with LGI1-IgM antibodies had relapses, compared to 1/104 without relapses (p<0.0001; Fisher's exact test). From the 7 relapsing patients with LGI1-IgM antibodies, these were detected within 4 weeks of the relapse in 5/7 cases. Only 2 of the 104 patients without LGI1-IgM antibodies had relapses (p<0.0001; Fisher's exact test).

This equates to a very high odds ratio (357; 27-3947) with >98% specificity and >99% negative predictive value of LGI1-IgM for a relapse:

| Odds ratio | 357.0 (26.82 to 3947) |
|---|---|
| Sensitivity | 0.8750 |
| Specificity | 0.9808 |
| Positive Predictive Value | 0.7778 |
| Negative Predictive Value | 0.9903 |

### Example 7—CXCL13 is Indicative of Relapse

The data discussed above demonstrate that antigen-specific IgMs are an effective surrogate measure of active germinal centre reactions. Another well-established marker of germinal centre activity is a cytokine (CXCL13), produced by cells which partake in germinal centre reactions, including follicular dendritic and T follicular helper cells.

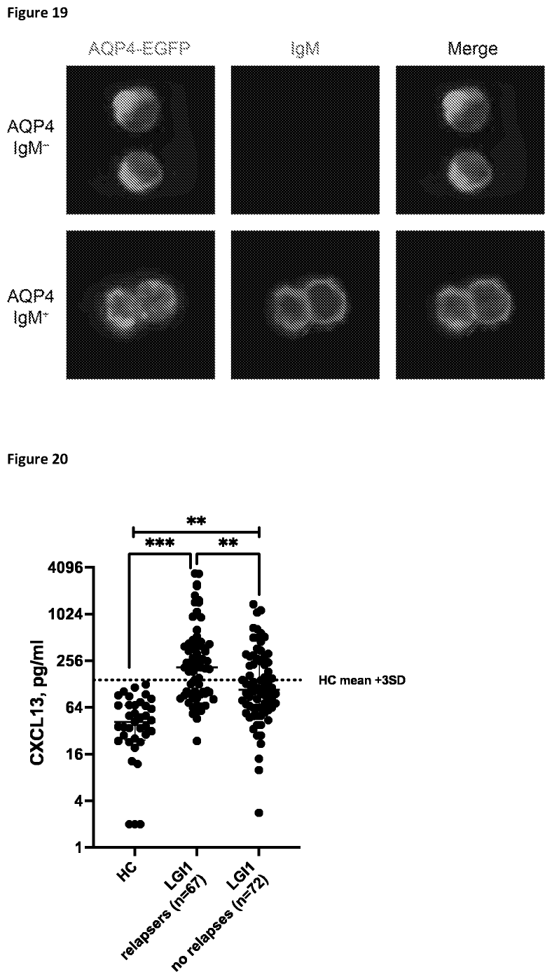

CXCL13 was therefore measured from a subset of patients/samples (142 serum samples of 48 LGI1-antibody patients). From 37 healthy control samples, a cut-off of mean plus 3 standard deviations was derived for CXCL13 levels (145 pg/ml). Based on this, from a larger cohort, 59/87 (68%) of positive CXCL13 samples were from relapsing patients and 59/84 (70%) of samples from relapsing patients were CXCL13 positive (both p<0.0001; Fisher's exact test; FIG. 20).

| | CXCL13 positive | CXCL13 negative |
|---|---|---|
| | (n = 87) | (n = 71) |
| Relapsing (n = 84) | 59 | 25 |
| Non-Relapsing (n = 74) | 28 | 46 |

Serum CXCL13 levels were significantly higher in sera from relapsing versus non-relapsing LGI1-autoantibody patients and relapsing patients more frequently showed CXCL13 levels above the cut-off (70% of sera versus 30% of sera from non-relapsing patients; p<0.0001).

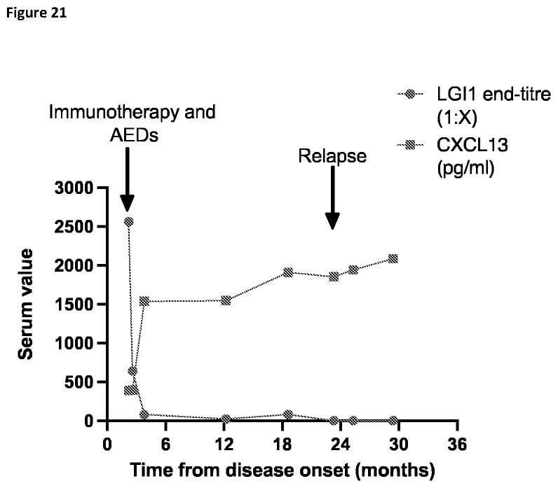

Further, in individual patients, persistently raised CXCL13 levels were observed, after LGI1-autoantibodies became very low or undetectable, particularly in patients with refractory disease (FIG. 21).

TABLE 1
Clinical and demographic characteristics of the two NMOSD
cohorts included in the study. Patients data were tested
for normality (Gauss distribution with the Anderson Darling,
D'Agostino & Pearson, Shapiro-Wilk and Kolmogorov-Smirnov
tests). Normally distributed data were analysed with an unpaired
t-test (*) and, otherwise, a Mann-Whitney U test (°).
AQP4-IgG = aquaporin 4; ARR = annualised relapse
rate; IgG = Immunoglobulin G; NMOSD = Neuromyelitis
Optica spectrum disorders; RTX = Rituximab

| | RTX | No RTX | Statistical |
|---|---|---|---|
| | (N = 35) | (N = 28) | comparisons |
| Female proportion | 30/35 (86%) | 25/28 (89%) | p = 0.48* |
| Mean age at NMOSD onset | 36 | 45 | p = 0.007* |
| Mean disease duration | 132 | 128 | p = 0.90* |
| (months) | | | |
| Median ARR pre-RTX | 0.89 | 0.28 | p < 0.001° |
| Median number of | 2 | 1 | p = 0.10° |
| immunotherapies pre-RTX | | | |
| Median AQP4-IgG titre pre- | 200 | 800 | p = 0.09° |
| RTX | | | |

TABLE 2
Clinical and demographic characteristics of the two NMOSD
cohorts included in the study. Patients data were tested
for normality (Gauss distribution with the Anderson Darling,
D'Agostino & Pearson, Shapiro-Wilk and Kolmogorov-Smirnov
tests). Normally distributed data were analysed with an unpaired
t-test (*) and, otherwise, a Mann-Whitney U test (°).
AQP4 = aquaporin 4; ARR = annualised relapse rate;
IgG = Immunoglobulin G; NMOSD = Neuromyelitis Optica
spectrum disorders; RTX = Rituximab.

| | RTX | No RTX | Statistical |
|---|---|---|---|
| | (N = 35) | (N = 28) | comparisons |
| Female proportion | 30/35 (86%) | 25/28 (89%) | p = 0.48* |
| Mean age at NMOSD onset | 36 | 45 | p = 0.007* |
| Mean disease duration | 132 | 128 | p = 0.90* |
| (months) | | | |
| Median ARR pre-RTX | 0.89 | 0.28 | p < 0.001° |
| Median number of | 2 | 1 | p = 0.10° |
| immunotherapies pre-RTX | | | |
| Median AQP4-IgG titre pre- | 200 | 800 | p = 0.09° |
| RTX | | | |

### Discussion

Sampling of blood from patients with NMOSD has revealed that switches in the dominant AQP4-IgG subclasses and AQP4-specific IgMs, especially cumulatively, associate with clinical relapses. These parameters could both be considered products of GC activity. dCLNs in patients with NMOSD has provided evidence of intranodal AQP4-specific B cells and intranodal synthesis of AQP4-IgGs, features which directly indicate GC activity as key to this AQP4-specific autoimmunization. As the present observations came from samples obtained at widely-varied timepoints over several years, they support the concept that AQP4-autoantibodies are generated by GC activity through the disease course. Of therapeutic importance, administration of RTX successfully depleted dCLN-resident B cells, indicating its potential therapeutic value in the disruption of GC activity. This putative mechanism of action may account for its rapid clinical efficacy despite limited effects in reducing serum AQP4-IgG levels. More broadly, this study presents an in vivo human paradigm to determine multiple biomarkers of GC activity, including valuable direct measures for clinical trials. In future, the translation of this paradigm may help better understand the biology of other autoimmune conditions and the mechanisms of action of other autoimmune therapeutics, and vaccinations for infectious diseases.³³

Alongside available studies, these observations identify many elements of a classical antigen-specific GC reaction in patients with NMOSD. Firstly, an early loss of B cell tolerance has been observed in NMOSD. This is consistent with the definitive isolation of unmutated, IgM-expressing, naïve AQP4-specific B cells from NMOSD PBMCs, and the successful construction of inferred mutation distance-based lineage trees between intrathecal AQP4-specific cells and peripheral double negative and naïve B cells. The unmutated naïve BCRs are likely to have a low affinity for AQP4, and may be the first lymphocytes to bind AQP4 epitopes in the initiation of GCs. During clinical relapses, the observed spikes in AQP4-IgM suggest GC activity is driven by the preferential recruitment of naïve B cells, rather than IgG⁺ memory B cell reactivation. Although IgM⁺ memory B cells, which are detected, may play a role, the rarity of memory B cell re-entry into GCs is emphasised by recent experimental murine data. Subsequently, it is likely that with help from AQP4-specific T cells, these naïve AQP4-specific B cells differentiate and mutate into higher-affinity AQP4-specific memory B cells, which are captured from both blood and dCLNs. The overall frequency of AQP4-specific B cells is far higher than most chronic anti-microbial responses, and suggests GCs in NMOSD patients are often occupied with the AQP4-directed autoimmunisation.

dCLNs are proposed as a plausible site for GCs in a disease driven by a CNS-predominant antigen, as they directly drain CNS lymphatics. dCLNs may be the first peripheral structures to encounter CNS-expressed AQP4. AQP4 antigen detection from human dCLN-based GCs is now a realistic aim, especially given blood contamination in aspirates appeared minimal. However, histological features of ectopic GCs have been described in the orbit of two patients with NMOSD, and it may be that CNS AQP4-rich sites are seeded with AQP4-B cells after their migration into the CNS.

Further still, the data demonstrates that LGI1-IgMs associate with >98% specificity and >99% negative predictive value for relapses, typically within 4 weeks of the attack. Hence, they also provide a specific predictive biomarker for a relapse. Their presence provides an incentive to consider increased immunosuppression for patients with LGI1-antibody encephalitis.

Another marker of germinal centre reactions (CXCL13) is also demonstrated to be raised in patients with LGI1-antibody encephalitis, especially patients with relapses. They may remain elevated in immunotherapy refractory patients once LGI1-antibodies are no longer elevated, hence providing a further independent and valuable biomarker, triggering the use of further immunosuppression. This approach would also predict a specifically increased potential utility for precision medicine with CXCL13-directed therapeutics.

In conclusion, this study demonstrates that the appearance of or increase in IgM antibodies, and/or that a change in the predominant IgG subclass, which recognise antigens known to be clinically relevant for a given antibody-mediated autoimmune disease, such as AQP4 in NMOSD, is a statistically significant indicator that a subject is in or will enter a relapse, and/or that a subject should be treated for the given antibody-mediated autoimmune disease. Specifically, the data suggests that the appearance or increase in AQP4-IgM, LGI1-IgM, or CXCL13, for example, during the disease course suggests that a patient has a high chance of relapsing, whilst if there is no detectable increase or presence of IgM antibodies or cytokine, a patient has a strong chance of not relapsing. The findings have implications for monitoring patients, evaluating the basis of treatment escalation and aim to directly appreciate the underlying disease biology. For example, identifying patients who are likely not in relapse removes the need for expensive treatment, and spares the patient the side effects of the conventional immunotherapies. GC-centric biomarkers can immediately enter the clinic and may be an avenue towards precision medicine for antibody-mediated autoimmune diseases.

1. A method of determining the disease status of a subject that has been diagnosed with an antibody-associated autoimmune disease or an antibody-mediated autoimmune disease wherein the antibody recognises a specific antigen, and wherein the method comprises:
a) providing a biological sample obtained from a subject; and
b) determining the level, or presence, of IgM isotype antibodies which recognise the specific antigen; and/or the level, or presence, of CXCL13.

2. The method of claim 1, wherein the disease status is: determining whether the subject is in a relapse, determining the likelihood of a relapse, determining whether the subject is to be treated, or determining whether a subject is likely to benefit from treatment.

3. The method of claim 1 or 2, wherein:
(a) the presence of IgM isotype antibodies which recognise the specific antigen indicates a higher likelihood of a relapse, or indicates that the subject is in a relapse, or indicates that the subject should be treated, or indicates that the subject is likely to benefit from treatment; or
(b) a higher level or no change in the level of IgM isotype antibodies which recognise the specific antigen in the biological sample obtained from a subject relative to the level of IgM isotype antibodies which recognise the specific antigen in a reference sample indicates a higher likelihood of a relapse, or indicates that the subject is in a relapse, or indicates that the subject should be treated, or indicates that the subject is likely to benefit from treatment; or
(c) the absence of IgM isotype antibodies which recognise the specific antigen indicates a lower likelihood of a relapse, or indicates that the subject is not in a relapse, or indicates that the subject should not be treated, or indicates that the subject is not likely to benefit from treatment; or
(d) a lower level of IgM isotype antibodies which recognise the specific antigen in the biological sample obtained from a subject relative to the level of IgM isotype antibodies which recognise the specific antigen in a reference sample indicates a lower likelihood of a relapse, or indicates that the subject is not in a relapse, or indicates that the subject should not be treated, or indicates that the subject is not likely to benefit from treatment.

4. The method of any of claims 1-3, wherein the method further comprises the step of determining the level of, or change in the level of, one or more IgA classes which recognise a specific antigen present in the biological sample obtained from the subject.

5. The method of any of claims 1-4, wherein the method further comprises the step of determining the change in predominant epitope recognised by a given Ig present in the biological sample obtained from the subject.

6. The method of any of claims 1-5, wherein the method further comprises the step of determining the level of, or change in the level of, one or more IgG subclasses which recognise a specific antigen present in the biological sample obtained from the subject; or
in place of determining the level or presence of IgM isotype antibodies which recognise the specific antigen, the method alternatively comprises the step of determining the level of, or change in the level of, one or more IgG subclasses which recognise a specific antigen present in the biological sample obtained from the subject; or
in place of determining the level or presence of IgM isotype antibodies which recognise the specific antigen, the method comprises the step of determining if there has been a switch in the predominant IgG subclass of the antibodies that recognise a specific antigen.

7. The method of claim 6, wherein an increase in the level of a given IgG subclass which recognise a specific antigen compared to the level of that IgG subclass which recognise the specific antigen in a reference sample indicates a higher likelihood of relapse, or indicates that the subject is in a relapse, or indicates that the subject should be treated, or indicates that the subject is likely to benefit from treatment; or
wherein when the level of a given IgG subclass which recognise a specific antigen does not increase compared to the level of that IgG subclass which recognise the specific antigen in a reference sample, indicates a lower likelihood of a relapse, or indicates that the subject is not in a relapse, or indicates that the subject should not be treated, or indicates that the subject is not likely to benefit from treatment.

8. The method of any of claim 6 or 7, wherein the IgG subclass is one or more of, such as all of IgG1, IgG2, IgG3, and/or IgG4.

9. The method of any of claims 1-8, wherein the antibody-mediated autoimmune disease is a Neuromyelitis optica spectrum disorder (NMOSD) or encephalitis.

10. The method of any of claims 1-9, wherein when the antibody-mediated autoimmune disease is NMOSD, the specific antigen is AQP4; and/or when the antibody-mediated autoimmune disease is encephalitis the specific antigen is LGI1, optionally wherein when the antibody-mediated autoimmune disease is encephalitis, the method further comprises determining the level, or presence, of CXCL13.

11. The method of any of claims 1-10, wherein when the antibody-mediated autoimmune disease is NMOSD, the treatment is one or more of an anti-CD20 antibody, such as Rituximab, Ofatumumab or Ocrelizumab; a steroid; azathioprine; mycophenolate mofetil; an agent, such as a mAb, which recognises IL-6R or reduces IL-6R activity; an agent, such as a mAb, which recognises CD19 or reduces CD19 activity; an agents which alter the function of complement pathway components (such as Eculizumab); and bortezomib; for example; and/or
when the antibody-mediated autoimmune disease is encephalitis, the treatment is with one or more immunotherapy such as with corticosteroids, intravenous immunoglobulins, plasma exchange, B cell depleting agents (e.g. rituximab (RTX) and inebilizumab), IgG depleting agents (e.g. FcRn inhibitors), cyclophosphamide, azathioprine, mycophenolate mofetil and satralizumab.

12. The method of any of claims 3-11, wherein the reference sample is blood, serum, plasma, saliva, lymph node aspirate such as deep cervical lymph node aspirate, or cerebrospinal fluid sample obtained from the subject.

13. The method of any of claims 3-12, wherein the reference sample is a lymph node aspirate such as deep cervical lymph node aspirate obtained from the subject.
